(19) United States
(12) Patent Application Publication
(10) Pub. No.: US 2026/0284262 A1
(43) Pub. Date: Sep. 24, 2026

(54) DECELLULARIZED, DELIPIDATED TISSUES, HYDROGELS THEREOF, AND USES THEREOF

(71) Applicant: University of Florida Research Foundation, Inc., Gainesville, FL (US)

(72) Inventors: Christine E. SCHMIDT, Gainesville, FL (US); Gopal AGARWAL, Gainesville, FL (US); Michaela W. MCRARY, Gainesville, FL (US)

(21) Appl. No.: 19/474,767

(22) PCT Filed: Apr. 16, 2024
(86) PCT No.: PCT/US2024/024739

§ 371 Date: Oct. 13, 2025

Related U.S. Application Data
(60) Provisional application No. 63/460,023, filed on Apr. 17, 2023.
(60) Provisional application No. 63/568,483, filed on Mar. 22, 2024.

(51) Int. Cl. A61L 27/36; A61L 27/52; A61L 27/54

(52) U.S. Cl. CPC A61L 27/3633; A61L 27/3604; A61L 27/3687; A61L 27/52; A61L 27/54; A61L 2430/22; A61L 2430/32; A61L 2430/40

(57) ABSTRACT

Described herein are decellularized, delipidated tissue hydrogels and hydrogel precursors and methods of producing decellularized, delipidated tissue hydrogels. In some aspects, the decellularized tissue hydrogels can contain one or more extracellular matrix proteins. Also described herein are methods of using the decellularized, delipidated tissue hydrogels. In some aspects, the decellularized, delipidated tissue hydrogels or a pre-gel solution can be administered to a subject.

### CROSS REFERENCE TO RELATED APPLICATIONS

This application claims the benefit of U.S. Provisional Application No. 63/460,023 filed on Apr. 17, 2023, and U.S. Provisional Application No. 63/568,483 filed on Mar. 22, 2024, both of which are incorporated herein by reference in their entireties.

### STATEMENT REGARDING FEDERALLY SPONSORED RESEARCH

This invention was made with government support under grant number R21 NS123596, awarded by the National Institutes of Health. The government has certain rights in this invention.

### BACKGROUND

In the United States alone, ~300,000 patients live with traumatic spinal cord injury (SCI). SCI can result from contusion, compression, laceration, or stretch of spinal cord tissue due to traumatic insult, which leads to permanent loss of function and sensation throughout the body. Presently, there is no effective treatment that results in functional recovery after SCI. Surgical decompression and methylprednisolone administration are the clinical standard-of-care for early acute SCI management, but they show no effect on functional recovery in SCI patients. Because of the lack of effective therapies to treat acute or subacute SCI, more than 95% of patients with SCI are in the chronic phase of injury, when glial scar is fully developed, and spontaneous behavioral recovery has reached its plateau. In human clinical trials, cell-based treatments for SCI are yet to provide reproducible evidence for clinical efficacy. As noted from preclinical studies, the primary reason for limited cell efficacy is due to poor cell viability and short residence at the SCI site. It would be desirable to produce an injectable hydrogel that increases Schwann cell efficacy by maintaining higher cellular viability during injection and increasing residence time post injection at the target site.

Autologous human Schwann cell transplantation is currently being investigated in U.S. Food and Drug Administration clinical trials for SCI patients with thoracic and cervical SCI. Phase I human clinical trials have demonstrated the feasibility and safety of transplantation of human Schwann cells with reduction in cyst volume. In rodent SCI models, transplanted Schwann cells secrete growth factors and extracellular matrix components such as collagen and laminin, ultimately providing a physical and bioactive substrate to promote axonal growth. Transplanted Schwann cells have also been shown to support new growth of sensory and propriospinal axons, myelinate regenerating or demyelinated axons, and restore axonal conduction. In addition, Schwann cell transplantation has been shown to improve functional recovery in preclinical subacute and chronic SCI models. However, direct local injection of Schwann cells into SCI results in significant transplanted cell loss and death, which limits the extent of the cells' therapeutic action. Previous studies have shown that after a contusion injury in rats, only ~20% of transplanted cells survive at 1 week and less than ~5% of Schwann cells survive 1 month after transplantation. The harsh SCI microenvironment with high levels of oxidative stress, inflammation, and immune response results in a low survival rate. In addition, typically up to ~60% of cells fail to even reach the target site, which is attributed to cell membrane damage during injection and cell reflux out of the spinal cord. Because Schwann cell retention has been correlated with decreased SCI cavitation and increased symptomatic relief, increasing the number of viable transplanted cells early after delivery would be expected to improve therapeutic efficacy.

Injectable hydrogels after in vivo injections at desired sites have the capacity to undergo sol-to-gel transition, resulting in an in situ solid hydrogel. Injectable hydrogels are in a flowable state before injection, allowing various water-soluble substances like drugs, proteins, cells, and genes to be easily loaded within the pre-gel injectable solution; thus, injectable hydrogels can be used as scaffolds or carriers for release of various therapeutic substances. Recently, injectable hydrogels have gained wide attraction, especially in biomedical applications, since injectability provides a way to deliver hydrogels with minimally invasive procedures.

Chemical and physical crosslinking strategies have been used widely for the fabrication of injectable hydrogels for SCI treatment. Unfortunately, chemical crosslinking of injectable hydrogels typically requires use of toxic crosslinking agents, which limits clinical translational potential. Physical gelation of hydrogels, on the other hand, is usually caused by changing intermolecular forces (e.g., hydrogen bonding, hydrophobic interactions, and electrostatic ionic forces) between different polymer chains, which can be achieved by changes in physical stimuli such as temperature, pH, ionic strength, or pressure. However, physically crosslinked hydrogels suffer from weak mechanical strength.

Decellularized extracellular matrix (dECM) has been widely used for over 20 years as regenerative biomaterial scaffold material because of its unique biological activity and excellent biocompatibility. Decellularized scaffolds are immunologically tolerated matrices that retain the native structure, tissue cell adhesion proteins, growth factors, and glycosaminoglycans to provide appropriate tissue remodeling in the host. Hydrogels derived from decellularized tissue matrix have gained wide attention because of their higher tunability, injectability, tunable mechanical properties, and ease for modification with bioactive molecules. Decellularized porcine-based hydrogels have been shown to promote peripheral nerve regeneration. However, in most studies, porcine-based dECM has been used as a filler or for coating of electrospun nanofibrous scaffolds. In addition, the potential of decellularized porcine peripheral nerve-based hydrogels as injectable delivery vehicles for cells or drugs is still unexplored.

Various chemical and enzyme-based decellularization methods have been used for porcine peripheral nerves. Also, other porcine tissues have been decellularized, but since each tissue has a different structure and composition, the decellularization methods are tissue specific. To keep tissue as intact as possible after decellularization, chemical methods need to be chosen carefully to minimize the damage caused to structural macromolecules of the ECM.

Tissue scaffolds, particularly hydrogel-based tissue scaffolds, play key roles in tissue engineering, repair of soft tissues, and can serve as drug delivery platforms. As such, there exists a need to generate useful tissue-based hydrogels and pre-hydrogel compositions. Previous methods of decellularization damaged valuable extracellular matrix (ECM) tissue components, such as certain ECM proteins. Other methods were developed to decellularize tissues while preserving extracellular matrix components, but for some tissue sources, the decellularized tissue exhibited poor gelation and/or could not form hydrogels at biologically relevant temperatures and were thus not suitable for clinical applications such as injectable formulations. There exists a need to generate useful tissue-based pre-hydrogel compositions and hydrogels capable of with desirable gelation kinetics.

### SUMMARY

Described herein are embodiments of a decellularized, delipidated tissue hydrogel that can be made of decellularized, delipidated tissue, wherein the decellularized, delipidated tissue can contain native extracellular matrix proteins and wherein the decellularized, delipidated tissue is cross-linked to form the hydrogel. The present disclosure also includes decellularized, delipidated tissue hydrogel precursor solutions (e.g., a pre-hydrogel solution).

In some embodiments, the decellularized, delipidated tissue hydrogels of the present disclosure can further include a therapeutic agent. The therapeutic agent can be a population of cells, a polynucleotide, an amino acid, a peptide, a polypeptide, an antibody, an aptamer, a ribozyme, a guide sequences for a ribozyme that inhibit translation or transcription of essential tumor proteins and genes, a hormone, an immunomodulator, an antipyretic, an anxiolytic, an antipsychotic, an analgesic, an antispasmodic, an anti-inflammatory agent, an anti-histamine, an anti-infective, a chemotherapeutic, or a combination thereof. In some embodiments, the therapeutic agent can also include extracellular vesicles and/or microRNA, such as described in Hernandez, et al. (Decellularized Extracellular Matrix Hydrogels as a Delivery Platform for MicroRNA and Extracellular Vesicle Therapeutics, Adv. Ther (Weinh) 2018 July; 1(3) 1800032), which is hereby incorporated by reference herein. The decellularized, delipidated tissue can be decellularized, delipidated brain, spinal cord, heart, lung, liver, muscle, cartilage, tendons, ligament, menisci, stomach, intestine, pancreas, kidney, blood vessel, bone, or cornea tissue.

The present disclosure also provides methods of making and using the decellularized, delipidated tissue hydrogel and pre-hydrogel solutions of the present disclosure.

In embodiments, the decellularized tissue hydrogel can be formed by the method that can include the steps of decellularization, delipidation, digestion, neutralization and then incubation at body temperature.

Also described herein are embodiments of a method that can include the step of injecting an amount of a pre-gel solution as described herein into a subject. In some embodiments, the method can include the step of co-administering a population of cells with the pre-gel solution of the present disclosure. In some embodiments, the subject can have a peripheral nerve injury, spinal cord injury, spinal cord disease, traumatic brain injury, amputation, degradation of a component of the spine, osteoarthritis, lung trauma, volumetric muscle loss, or liver cirrhosis.

### BRIEF DESCRIPTION OF THE DRAWINGS

Further aspects of the present disclosure will be readily appreciated upon review of the detailed description of its various embodiments, described below, when taken in conjunction with the accompanying drawings.

Further aspects of the present disclosure will be readily appreciated upon review of the detailed description of its various embodiments, described below, when taken in conjunction with the accompanying drawings.

FIGS. 1A-1D are flow charts illustrating embodiments of methods of the present disclosure for producing decellularized, delipidated tissue hydrogels and hydrogel precursors. FIG. 1A shows a flow diagram demonstrating an embodiment of method steps to form a decellularized, delipidated tissue hydrogel from the decellularized tissues made from the decellularization and delipidation methods described herein. FIGS. 1B-1Cii illustrate flow diagrams demonstrating general steps in an apoptosis-based methods (FIG. 1B) and sodium deoxycholate-deoxyribonuclease (SDD) methods (FIGS. 1Ci-1Cii) of chemical decellularization method that can result in an acellular tissue product that retains one or more extracellular matrix components, which can then be used in the process illustrated in the flow chart of FIG. 1D for delipidation of the decellularized tissue from FIG. 1B or FIGS. 1Ci-1Cii to produce a decellularized, delipidated tissue hydrogel.

FIGS. 2A-2D are images illustrating the efficiency of an embodiment of an SDD decellularization method for removal of cellular and nuclear debris from porcine nerve tissues (sciatic (FIG. 2A), tibial (FIG. 2B), peroneal (FIG. 2C), sural (FIG. 2D)).

FIGS. 3A-3E are images illustrating preservation of extracellular matrix components in various porcine peripheral nervous tissues (sciatic (FIG. 3A), tibial (FIG. 3B), peroneal (FIG. 3C), sural (FIG. 3D)) after decellularization. FIG. 3E shows a close-up view of the fresh vs. decellularized tissues.

The graphs in FIGS. 4A-4D show estimation of lipid content using vanillin reagent in decellularized porcine nerves (sciatic (FIG. 4A), tibial (FIG. 4B), peroneal (FIG. 4C), sural (FIG. 4D)) before and after an embodiment of a delipidation method of the present disclosure. Non-delipid nerves are also decellularized, but not treated with lipid removal solvent.

FIGS. 5A-5D are graphs illustrating UV spectrophotometer analysis of the gelation kinetics of decellularized and delipidated peripheral nerves (sciatic (FIG. 5A), tibial (FIG. 5B), peroneal (FIG. 5C), sural (FIG. 5D)) in embodiments of pre-gel solutions of the present disclosure. A control sample containing only decellularized nerves had no significant increase in absorbance value over time and was assigned a negative value during calculations.

FIGS. 6A-6I are graphs illustrating thermal gelation kinetics of pre-gel solutions prepared from decellularized and delipidated peripheral nerve: peroneal (FIGS. 6A-6C); sciatic (FIGS. 6D-6F); tibial (FIGS. 6G-6I).

FIG. 7 shows immunostaining images representing efficient cell removal using SDD method: DAPI, β-tubulin III, S100, and MBP staining to analyze the removal of various cellular contents in decellularized nerves. (Scale bar=50 μm).

FIG. 8 shows immunostaining images representing efficient ECM retention in decellularized nerves: Col-IV, Col-I and laminin staining to analyze the ECM structure maintenance in decellularized nerves (Scale bar=50 μm).

FIGS. 9A-9B show temperature sweep studies of (FIG. 9A) sciatic decell+delipid and decell only (FIG. 9B) peroneal sciatic decell+delipid and decell only.

FIGS. 10A-10F show genipin crosslinking tunes mechanical strength of hydrogels and affects injectability and gelation kinetics: Effect of genipin-mediated crosslinking on decell+delipid hydrogels, (FIG. 10A) sciatic (FIG. 10B) peroneal. Data were analyzed using One Way ANOVA Tukey t test, n=3. Time sweep experiment to analyze the effect of genipin mediated crosslinking on decell+delipid hydrogel, (FIG. 10C) sciatic (FIG. 10D) peroneal. Shear thinning experiment to demonstrate injectability of decell+delipid, (FIG. 10E) sciatic (FIG. 10F) peroneal.

FIGS. 11A-11C show injectable hydrogel maintains Schwann cell viability postinjection: (FIG. 11A) Schematic representative of overall experiment performed: Schwann cells encapsulated within injectable hydrogel formulations were passed through 25-G needles fitted into 1 mL syringes. The cells were stained with live/dead stain and imaged using confocal microscopy immediately after 30 minutes and at days 4 and 7. (FIG. 11B) Representative confocal images of live and dead stained cells after 30 minutes and days 4 after passing through the 25-G needle. Scale bar=200 μm. (FIG. 11C) Bar graph showing live/dead ratio of Schwann cells, analyzed using Image J software. Data were analyzed using Two Way ANOVA, Bonferroni test, compared vs saline control, n=3. ***p<0.0001.

FIGS. 12A-12D show genipin-crosslinked hydrogel maintains Schwann cell viability in an in vitro inflammatory model: (FIG. 12A) Cartoon showing astrocyte-Schwann cell co-culture experiment with LPS as in vitro inflammatory model (FIG. 12B) representative image showing GFAP intensity in LPS and non-treated astrocytes (FIG. 12C) live/dead staining images of Schwann cells after 48-hour incubation in the presence of LPS inflamed astrocytes; live cells are and dead cells are represented by differential staining. TCP: tissue culture plastic seeded Schwann cells (FIG. 12D) live/dead cell ratio as analyzed by Image J software. Data are analyzed by One Way ANOVA, Tukey t test, n=3.

FIG. 13 shows live/dead staining of Schwann cells post injection: Representative confocal images of live and dead Schwann cells stained at day 7 post injection. Scale bar=200 μm.

FIGS. 14A-14B show the effect of different concentrations of LPS on astrocyte reactivity: (FIG. 14A) Representative images of GFAP/DAPI stained confocal images. (FIG. 14B) Image J based GFAP/DAPI intensity analysis. Data are analyzed using One way ANOVA, Tukey t test, n=3.

FIG. 15 shows percent degradation of uncrosslinked and crosslinked sciatic and peroneal decell+delipid hydrogels after 4 days and after 14 days. Uncrosslinked hydrogels degrade faster than crosslinked hydrogels, with more degradation on day 14 than on day 4.

FIG. 16 shows a flow chart showing a process for producing injectable hydrogel formulations using decellularized human peripheral nerves.

FIGS. 17A-17C show decellularized nerves subjected to different delipidation procedures. FIG. 17A: decell only. FIG. 17B: decell+delipid using dichloromethane/ethanol mixture. FIG. 17C: decell+delipid using n-hexane/isopropanol mixture.

FIGS. 18A-18C show Collagen-I ECM and DAPI staining of human nerve tissue subjected to different treatments. FIG. 18A: fresh human nerve. FIG. 18B: decell human nerve. FIG. 18C: decell+delipid human nerve (using dichloromethane:ethanol in a 2:1 v/v ratio).

FIGS. 19A-19D show Oil Red O staining of human nerve tissue subjected to different treatments. FIG. 19A: fresh human nerve. FIG. 10\9B: decell human nerve. FIG. 19C: decell+delipid human nerve (using dichloromethane:ethanol in a 2:1 v/v ratio). FIG. 19D: decell+delipid human nerve (using hexane:isopropanol in a 3:2 v/v ratio).

FIG. 20A shows human decell+delipid nerves digested using pepsin, neutralized using 1 N NaOH and 10×PBS, crosslinked using 5 mM genipin, cast into a silicone mold, and incubated at 37° C. for 15-20 min. FIG. 20B shows a frequency sweep rheology study of a hydrogel prepared as in FIG. 20A. Mechanical strength (storage modulus) is shown in the top, decreasing line, while loss modulus is shown in the lower, increasing line.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used in the specification and the appended claims, the singular forms “a,” “an,” and “the” include plural referents unless the context clearly dictates otherwise.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible unless the context clearly dictates otherwise.

### Definitions

As used herein, “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes but is not limited to, subcutaneous, intravenous, intramuscular, intra-articular, intra-synovial, intrasternal, intrathecal, intrahepatic, intralesional, and intracranial injections or infusion techniques.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “control” is an alternative subject or sample used in an experiment for comparison purposes and included to minimize or distinguish the effect of variables other than an independent variable.

As used herein, “positive control” refers to a “control” that is designed to produce the desired result, provided that all reagents are functioning properly and that the experiment is properly conducted.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “autologous” refers to being derived from the same subject that is the recipient.

As used herein, “allograft” refers to a graft that is derived from one member of a species and grafted in a genetically dissimilar member of the same species.

As used herein “xenograft” or “xenogeneic” refers to a substance or graft that is derived from one member of a species and grafted or used in a member of a different species.

As used herein, “autograft” refers to a graft that is derived from a subject and grafted into the same subject from which the graft was derived.

As used herein, “allogeneic” refers to involving, derived from, or being individuals of the same species that are sufficiently genetically different so as to interact with one another antigenically.

As used herein, “donor” refers to a subject from which cells or tissues are derived.

As used herein, “hypertonic solution” refers to a solution that has a greater concentration of solutes than the concentration of solutes inside of a cell.

As used herein, “hypotonic solution” refers to a solution that has a concentration of solutes that is less than the concentration of solutes within a cell.

As used herein, “analog” refers to another compound, composition, or other substance of matter that is considered comparable to the reference compound, composition, or substance of matter and includes structural analogs and functional analogs.

### Discussion

Decellularization technology has provided the potential to attain tissue-specific scaffolds that guide tissue regeneration following injury and/or disease. However, most decellularization protocols used in research and industry contain an initial cell lysis step in a hypotonic buffer, such as water, followed by harsh chemical solutions to remove cellular remnants. These harsh conditions result in an undesirable broad dispersal of intracellular components, disruption of tissue morphology, and removal of desired tissue elements.

With that said, decellularization methods have been developed that retain desirable tissue elements, such as ECM and have less disruption of tissue morphology. However, while tissues from some animal sources could be decellularized by such methods and made into injectable hydrogels without further modification, some tissues (such as, but not limited to, human and porcine never tissues) would not form hydrogels without modification and/or gelation at high temperatures, rendering them unsuitable for injectable formulations to form hydrogel in situ. Thus, it was found that, depending on the tissue source, the pre-hydrogel solutions produced by some of these methods did not have desirable gelation kinetics for use as injectable formulations. As described in greater detail in the Example below, through a series of studies it was discovered that the presence of a greater percentage of lipids in some tissues was inhibiting gelation and that removing the lipids from the decellularized tissue while preserving the ECM components improved the gelation kinetics. It was found that the formulations prepared from the decellularized, delipidated tissues can be injected at a site of injury and will gel to form hydrogels in situ at body temperature and that conform to the injury space. The decellularized, delipidated materials include extracellular matrix (ECM) components that provide cues for regenerating tissues (such as nerve and other tissues).

Thus, the present disclosure provides decellularized, delipidated tissue hydrogels, as well as pre-hydrogel compositions and methods of making and using the tissue hydrogels and pre-hydrogel compositions. Embodiments of the present disclosure provide injectable, decellularized, delipidated, tissue hydrogels with ECM protein composition and mechanical properties that mimic native tissues which can be used for tissue repair. In embodiments the tissue hydrogel is prepared from nerve tissue and can be used for neural regeneration such as for use to repair tissue in situations such as, but not limited to peripheral nerve injury, spinal cord injury, and/or brain injury). In addition, the tissue hydrogels and pre-hydrogel solutions of the present disclosure can be used in a minimally invasive manner, which is beneficial to improve outcome, minimize cost, and reduce the potential for further damage during a major surgical procedure.

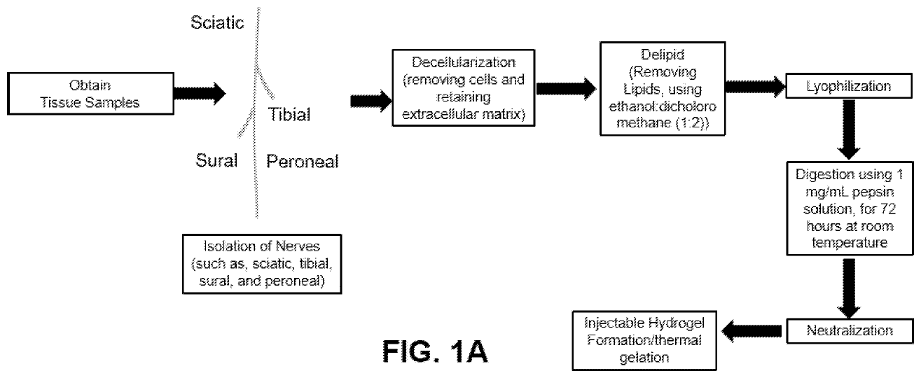

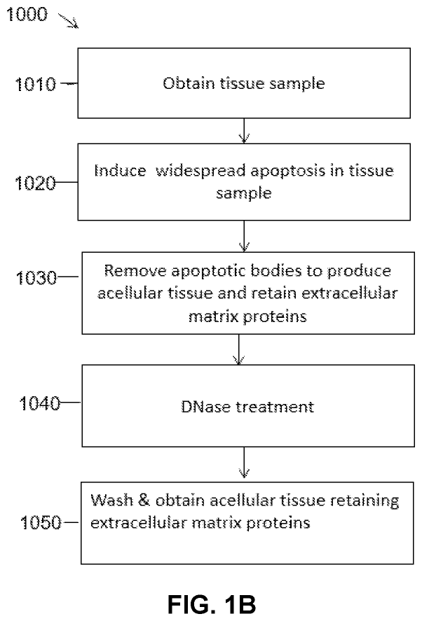

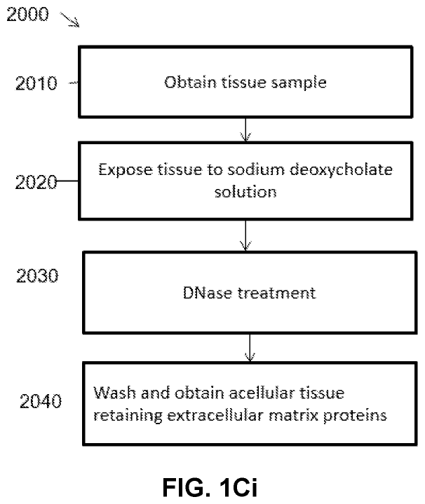

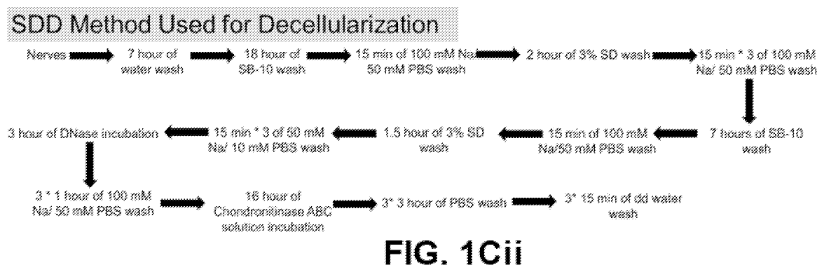

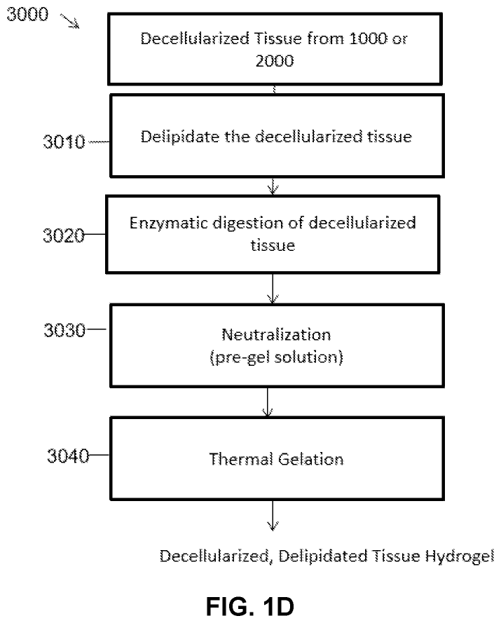

As illustrated in FIG. 1A, embodiments of the present disclosure can include obtaining tissue samples from a subject (e.g., mammal such as, but not limited to, a rat, bovine, pig, human, etc.). In an embodiment where nervous tissue scaffolds and/or gels are desired, nerve tissue is obtained and nerves can be isolated, such as shown in FIG. 1A. The isolated tissue is then subject to decellularization as described herein (such as, for instance, via apoptosis or chemical methods) to remove cells while retaining extracellular matrix components. The tissue can also be subject to removal of residual genetic material, such as DNA, such as for instance, by treatment with DNase as described herein. As shown in FIG. 1A, after decellularization and optional washing step, the tissue can be treated as described in greater detail below to remove residual lipids to provide improved gelation kinetics. In embodiments, the decellularized, delipidated tissue can be lyophilized for preservation until use. The fresh or lyophilized tissue can then be digested as described herein. As digestion generally involves acidic conditions, the digested tissue can then be neutralized to a biologically acceptable pH, such as a neutral pH of about 7.0 to about 7.6, e.g., about 7.3. After neutralization a decellularized, delipidated tissue pre-gel solution with native ECM components is obtained. This pre-gel solution can be combined with other additives as described below (such as crosslinkers, cell populations, therapeutics, growth factors, and combinations thereof). The pre-gel solution can be subject to thermal hydrogel formation, which occurs at a near biological body temperature and can thus occur after injection in vivo for a minimally invasive procedure or can be induced ex vivo for preparation of an implantable scaffold. Some of such steps are illustrated in FIG. 1A but will be described in greater detail in the description and example below.

The decellularization methods described herein can effectively remove cellular components without having to rely on harsh conditions. In some embodiments, the methods can effectively remove cellular components from peripheral nerve and/or nucleus pulposus. Decellularization methods useful in the methods of the present disclosure can include those described in McCrary et al, Tissue Engineering Part C: Methods, 2020, 23-36 and International Patent Application publication number WO 2019/165103A1, which are hereby entirely incorporated by reference herein. In addition to reducing time, labor, and expense of decellularization, some embodiments described herein can only need one step for inducing apoptosis and one step for washing the cells. The tissues are also subject to delipidation to improve gelation kinetics. The delipidation process also does not subject the tissue to harsh conditions and retains the ECM components.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Apoptosis-Based Tissue Decellularization Methods

Described herein are methods of decellularization that include inducing apoptosis in an ex vivo tissue sample, where tissue-wide apoptosis can be induced by exposing the tissue to an apoptosis-inducing agent or apoptosis inducing process for a time period. Induction of apoptosis in the ex vivo tissue sample can cause cell detachment from the tissue extracellular matrix, degradation of intracellular DNA, RNA and proteins, and allocation into apoptotic bodies, which can be removed using mild hypotonic and/or mild hypertonic buffered solutions. Such methods are described in Patent Application publication number WO 2019/165103A1, incorporated above, and described briefly below.

As shown in FIG. 1B, the apoptotic decellularization method 1000 can begin with obtaining a tissue sample from a subject or other donor source 1010. The tissue can be autologous, xenogeneic, allogeneic, or syngeneic. In some embodiments, the tissue can be obtained from anywhere in a subject. In some embodiments, the tissue sample can be obtained from the periphery or spinal column of a subject. In some embodiments, the tissue can be a peripheral nerve and/or nucleus pulposus. In other embodiments, the tissue can be brain, spinal cord, heart, lung, liver, muscle, cartilage, tendons, ligaments, menisci, stomach, intestine, pancreas, and/or kidney. In further embodiments, the tissue is blood vessels, bone, and/or cornea. The amount of tissue can range from about 0.1 μg to 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 2.75 mg, 5 mg, 5.25 mg, 5.5 mg. 5.75 mg, 6 mg, 6.25 mg, 6.5 mg, 6.75 mg 7 mg, 7.25 mg, 7.5 mg, 7.75 mg. 8 mg, 8.25 mg, 8.5 mg, 8.75 mg, 9 mg, 9.25 mg, 9.5 mg, 9.75 mg, 10 mg, 11 mg, 12 mg, 13 mg, 14 mg, 25 mg, 16 mg, 17 mg, 18, mg, 19, mg, 20 mg, or more

After the tissue sample has been obtained, apoptosis can be induced 1020 in the ex vivo tissue sample by exposing the ex vivo tissue sample to an apoptotic agent (i.e., a compound and/or composition that induces apoptosis in a cell) for a time period. In some embodiments, apoptosis can be induced by an apoptotic process (i.e., a process that can include one or more steps that induces apoptosis). In some embodiments, the apoptotic agent can be camptothecin, staurosporine, doxorubicin, and/or analogs thereof, or any other agent or process that induces apoptosis. In other embodiments, the apoptotic agent could be nitric oxide, hypoxia, pH, or hydrogen peroxide. In some embodiments where an apoptotic agent is used to induce apoptosis, the ex vivo tissue sample can be exposed to the apoptotic agent for any length of time, including but not limited to, greater than about 1 hour, about 1 hour to about 10 days, about 1 hour to about 7 days, about 1 hour to about 4 days, about 1 hour to about 2 days, or about 1 hour to about 1 day. In some embodiments, the ex vivo tissue sample can be exposed to camptothecin or analog thereof for 2 days. In some embodiments, the tissue ex vivo tissue sample can be exposed to the apoptotic agent, such as camptothecin, for about 1 day.

The ex vivo tissue sample can be exposed to a solution containing a concentration of the apoptotic agent and/or to an apoptotic process. In some embodiments, the apoptotic process does not use an apoptotic agent. In embodiments that employ an apoptotic agent, the concentration of the apoptotic agent can range from about 1 nM to about 1 mM or greater. In some embodiments, the concentration of the apoptotic agent can range from about 1 μM to about 100 μM. In some embodiments, the concentration of the apoptotic agent can be about 10 μM. In some embodiments, the ex vivo tissue sample can be exposed to 10 μM of camptothecin or an analog thereof. In some embodiments, the ex vivo tissue sample can be exposed to 10 μM of camptothecin or an analog thereof for about 2 days. In some embodiments, the concentration of the apoptotic agent can range from about 5 μM to about 10 μM. In some embodiments, the concentration of the apoptotic agent is about 5 μM. In some embodiments, the ex vivo tissue sample can be exposed to 5 μM camptothecin or an analog thereof. In some embodiments, the ex vivo tissue sample can be exposed to 5 μM of camptothecin or an analog thereof for about 1 day.

In some embodiments, the apoptotic process includes exposure of the ex vivo tissue to one or more freeze-thaw cycles to induce apoptosis.

After the ex vivo tissue sample can be exposed to an apoptotic agent or process for a desired amount of time, the tissue sample can be washed 1030 with one or more washes with one or more buffered solutions to facilitate cell removal. The ex vivo tissue sample can be washed with one or more washes of a hypertonic buffered solution (i.e. greater than 1× buffered solution). In some embodiments the number of washes ranges from one to six. In some embodiments, the hypertonic solution can be a 1.01×-10× buffered solution. In some embodiments, the concentration can be 4× with an intermediate 2× wash. The tissue sample can be washed with one or more washes of a hypotonic buffered solution (i.e. less than 1× buffered solution). In some embodiments, the hypotonic solution can be a 0.01× up to a 0.99× buffered solution. In embodiments, the buffered solution is a saline solution at the given concentrations to result in a hypotonic or hypertonic solution. The starting saline solution can be isotonic. The starting saline solution can have a formulation of 10× which is diluted with water to attain various hypertonic washes. While not being bound to theory, it is believed that DNA removal occurs under the hypotonic wash conditions, while protein removal occurs under the hypertonic wash conditions. Some embodiments employ only hypertonic washes. Other embodiments employ only hypotonic washes. Yet further embodiments employ both hypertonic and hypotonic washes.

In some embodiments, the method 1000 can also include an optional step of DNAse treatment 1040. The DNAse treatment can occur after the wash(es) previously performed 1030. In some embodiments, the optional DNAse treatment 1040 is performed after hypotonic washes 1030 were performed. In some embodiments where the optional DNAse 1040 treatment is performed, only hypertonic washes have been performed. In embodiments, the concentration of DNAse can range from 25 U/mL to 250 U/mL. The preferred concentration is 75 U/mL. The DNase can be any suitable DNase. The DNase treatment can be performed at a temperature ranging from about 4° to about 37° C. The preferred temperature is 25° C. It will be appreciated by those of ordinary skill in the art that the temperature can be dependent on the temperature that the DNase has optimal activity. The time period for the DNase treatment can range from about 30 minutes or less to 1, 2, 3, 4, 5, 6, 7, 8, 9, 10, 11, 12, 24, or 36 hours or more. The treatment time can vary based on tissue type, but in some embodiments the treatment time is 18-24 hours.

After the optional DNase treatment or prior wash(es), the resulting decellularized tissue (also referred to herein as an acellular tissue product or scaffold) can be optionally washed. The decellularized tissue can be immediately used (e.g., implanted) or can be lyophilized or otherwise stored for later processing or use. In some embodiments, the resulting decellularized tissue can undergo thermal gelation to form a hydrogel. This is discussed in greater detail elsewhere herein.

The method 1000 can produce an acellular tissue. According to methods of the present disclosure, the resulting acellular tissue can be further subject to delipidation and used as a scaffold substrate for three-dimensional (3D) cell culture and tissue engineering techniques. The acellular, delipidated tissue can contain extracellular matrix proteins, including laminin and collagen. Further, the resulting acellular, delipidated tissue can be delivered to a subject in need thereof to facilitate the in vivo generation and/or regeneration of cells and tissues within the subject. The resulting acellular, delipidated tissue product can be formulated to be delivered to a subject in need thereof by any suitable method of deliver, including but not limited to, implantation or injection. The acellular, delipidated tissue products can be enhanced by the addition of one or more other agents or compounds to the acellular tissue product, including without limitation other cells (e.g. stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. In some embodiments, the acellular tissue products can be co-administered with one or more other agents or compounds, including without limitation other cells (e.g. stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

In some embodiments, any of the acellular, delipidated tissue scaffolds described herein can be administered to a subject in need thereof in the original form, as an injectable formulation, or as other derivatives of the acellular tissue. In some embodiments, the subject in need thereof has a peripheral nerve injury, spinal cord injury, spinal cord disease, amputation or otherwise suffers from degradation of a component of the spine (e.g., intervertebral disc degeneration). In other embodiments, the subject could have osteoarthritis of the hip or knee, trauma to the lungs, volumetric muscle loss, cirrhosis of the liver, or otherwise requires partial or total organ replacement.

### Chemically-Based Tissue Decellularization Methods

As discussed above, conventional harsh chemical processing of tissues used to decellularize tissue can strip the tissue completely of extracellular matrix proteins. Described herein are chemical decellularization methods that can produce an acellular tissue scaffold that retains one or more extracellular matrix proteins, including laminin, collagen (e.g. at least collagen-IV and -I). Such methods are also described in Patent Application publication number WO 2019/165103A1, incorporated above, and described briefly below.

FIGS. 1Ci-1Cii show flow diagrams demonstrating the general process for a chemical decellularization process 2000 that can generate an acellular tissue scaffold that retains extracellular matrix proteins, including collagen and laminin. Generally, an amount of tissue can be obtained 2010. The tissue sample can be obtained from a subject or other donor source. The tissue can be autologous, xenogeneic, allogeneic, or syngeneic. In some embodiments, the tissue can be obtained from anywhere in a subject. In some embodiments, the tissue sample can be obtained from the periphery or spinal column of a subject. In some embodiments, the tissue can be a peripheral nerve and/or nucleus pulposus. In other embodiments, the tissue can be brain, spinal cord, heart, lung, liver, muscle, cartilage, tendons, ligaments, menisci, stomach, intestine, pancreas, and/or kidney. In further embodiments, the tissue is blood vessels, bone, and/or cornea.

The tissue can be washed after initial harvesting. In some embodiments the initial wash can be in a solution containing sodium deoxycholate 2020. The percent sodium deoxycholate (SD) in the solution can be about 3% (w/v). In some embodiments the percent sodium deoxycholate can be about 2-5% (w/v.) The incubation in the SD solution can be about 2 hours. In some embodiments, the SD solution can contain a saline buffer and/or an amount of sulfobetaine 16. In some embodiments, the concentration of sulfobetaine-16 can be about 0.6 mM. In some embodiments, the saline buffer is a sodium/phosphate buffer. In some embodiments, the sodium/phosphate buffer can contain about 50 mM sodium. In some embodiments, the sodium/phosphate buffer can contain about 10 mM phosphate buffer. In some embodiments, the saline buffer contains about 50 mM sodium, about 10 mM phosphate, and about 0.6 mM sulfobetaine-16. The amount of tissue incubated in SD solution can range from about 0.1 μg to 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 2.75 mg, 5 mg, 5.25 mg, 5.5 mg. 5.75 mg, 6 mg, 6.25 mg, 6.5 mg, 6.75 mg 7 mg, 7.25 mg, 7.5 mg, 7.75 mg. 8 mg, 8.25 mg, 8.5 mg, 8.75 mg, 9 mg, 9.25 mg, 9.5 mg, 9.75 mg, 10 mg, 11 mg, 12 mg, 13 mg, 14 mg, 25 mg, 16 mg, 17 mg, 18, mg, 19, mg, 20 mg, or more. In some embodiments, the harvested tissue can be optionally initially washed prior to exposure to the solution containing SD. In some embodiments, the optional additional washes can be performed in a solution of water, phosphate buffered solutions (50 mM Na/10 mM phosphate, 1×PBS, or 100 mM Na/50 mM Phosphate), and 125 mM sulfobetaine-10. Wash times for SB-10 solution for nerve are 18 hours and 7 hours. The buffered washes can range in time from 15 minutes to 3 hours.

The tissue can be optionally washed in a saline buffer. After any optional washing, DNA can be removed by treatment with DNase 2030. DNase treatment can include incubation in a solution containing DNase. In some embodiments, the amount of DNAse can be at least 2 mL of a 75 U/mL DNAse solution. In some embodiments, the DNAse solution can contain at least 37 U DNase/mg of tissue. In some embodiments, the solution can contain an amount of DNase that can range from about 37 U/mg of tissue to 40, 45, 50, 55 60, 65, 70, 75. 80. 85, 90, 95, or 100 U/mg of tissue. In addition to the DNAse, the DNAse solution can also contain an amount of NaCl, and an amount of MgCl₂(6H₂O). After DNase treatment 2030, the tissue can be washed to yield the acellular tissue scaffold. The post-DNase treatment wash step can include three 1-hour washes. The wash solution can be water, PBS, or other saline buffered solution. In some embodiments, the PBS can contain 50 mM Na/10 mM phosphate, 1×PBS, or 100 mM Na/50 mM Phosphate. In some embodiments, the wash solution can contain SB-10. In other embodiments, the wash solution does not contain SB-10. In some embodiments, the amount of SB-10 can be about 125 mM. In some embodiments, the wash step can include 1 or more washes in the wash solution. The time for each wash can range from 15 minutes to 3, 7, or about 18 hours. When multiple washes are used, the solution for each wash step can be independently selected. In other words, some or all of the steps can use the same wash solution or some or all of the wash steps can use different wash solutions. Similarly, the time for each wash step can be independently selected. In other words, some or all of the washes can be performed for the same amount of time or some or all of the washes can be performed for different number of times. The wash steps can be performed with or without agitation.

The resulting decellularized tissue (also referred to herein as an acellular tissue product or scaffold) can then be subject to delipidation as described herein, and used (e.g. implanted) or can be lyophilized, chilled frozen, and/or otherwise stored for later processing or use. In some embodiments, the resulting decellularized, delipidated tissue can undergo thermal gelation to form a hydrogel. This is discussed in greater detail elsewhere herein.

The method 2000 can produce an acellular tissue. The resulting acellular tissue can be delipidated as described in FIG. 1D (3000) and FIG. 1A and used as described herein as a scaffold substrate for three-dimensional (3D) cell culture and tissue engineering techniques.

### Delipidation Methods

As discussed above, conventional harsh chemical processing of tissues used to decellularize tissue can strip the tissue completely of extracellular matrix proteins. While decellularization methods that preserve the ECM proteins are described above, it was found that even after such decellularization, some tissues were unable to effectively form hydrogels at relevant biological temperatures (such as standard body temperature). As described above, it was discovered that the gelation problems could be caused by residual lipid content on the tissues after decellularization. Thus, methods were developed to delipidate the tissues to remove residual lipids while still preserving useful ECM components. Described herein are chemical delipidation methods that can produce an acellular, delipidated tissue scaffold, pre-gel solution, and/or hydrogel that retains one or more extracellular matrix proteins, including laminin, collagen (e.g., at least collagen-IV and -I).

FIG. 1D shows a flow diagram demonstrating the general process for a process 3000 for delipidation and further processing of the decellularized tissue from 1000 or 2000 to generate an acellular tissue hydrogel pre-gel solution and tissue hydrogel matrix that retains extracellular matrix proteins, including but not limited to collagen and laminin.

Generally, the decellularized tissue from 1000 or 2000 can be obtained and washed as needed. The decellularized tissue is then subject to delipidation in 3010. Although the descriptions in FIGS. 1A-1D and the discussion here generally describe decellularization of the tissue as preceding delipidation in some embodiments, and with some delipidation solvents (such as Type I water), the delipidation step could possibly be carried out prior to or contemporaneously with decellularization. While the methods of the present disclosure are described with decellularization typically occurring prior to delipidation, it is the intention that methods of the present disclosure in which delipidation occurs before or simultaneously with decellularization include also be considered within the scope of the present disclosure. Delipidation can be achieved as described herein and the examples below with a process that retains the native eCM components to produce a decellularized, delipidated tissue.

In embodiments the decellularized tissue is incubated with a delipidation solvent solution for an amount of time to removed lipids. In embodiments, the delipidation solvent is a solvent that effectively removes lipids and reduces the overall amount of residual lipids from decellularized tissues, but does not damage the underlying decellularized tissue scaffold and preserves the ability of the tissue to form a hydrogel after digestion. In embodiments, the delipidation solvent is selected from solvents such as, but not limited to, a combination of ethanol and dichloromethane, methanol and dichloromethane, other alcohols, and type I water only. In embodiments, the ethanol and dichloromethane (or methanol and dichloromethane) are in a ratio of about 1:1 to about 1:4, such as about 1:2. In embodiments the tissue is incubated with the delipidation solvent for about 12 hours to about 72 hour, such as about 24 hours. In embodiments, the delipidation process is carried out at a temperature of about 4° C. to about 25° C., taking care to keep the temperature below about 32-37° C., to reduce the possibility of any premature gelation/crosslinking.

In embodiments, after delipidation the decellularized, delipidated tissue can optionally lyophilized for storage until use. As described below, when desired to be used, the optionally lyophilized decellularized, delipidated tissue can be enzymatically digested (3020). After digestion (e.g., with pepsin or other known method such as described herein), the tissue can then be neutralized to a biologically compatible pH prior to use. In embodiments, during digestion and neutralization, the decellularized, delipidated tissue is kept at a temperature of about 4° C. to 25° C. (e.g., below about 30° C.) so that the pre-gel solution does not begin to prematurely gel.

The neutralized, digested, delipidated, decellularized tissue can be used as a pre-gel formula and the hydrogel can be formed by raising the temperature to induce gelation either ex vivo or in vivo. Also, in embodiments, crosslinking agents can be added to the pre-gel solution to increase gelation speed to reduce the time to formation of the hydrogel. In embodiments, crosslinking agents can include, but are not limited to genipin, glutaraldehyde, dicyclohexylcarbodiimide (DCC) or ethyl(dimethylaminopropyl) carbodiimide (EDC), N-Hydroxysuccimide, sulfo-N hydroxysuccimide, 4S arm PEG Succinimidyl Glutarate, 4arm PEG Succinimidyl Carboxymethyl Ester or combination of these. Some such crosslinkers may not be suitable for use in vivo, but can be used if gel is formed in vitro to provide tissue scaffolds and/or for magnetic templating.

The acellular, delipidated tissue can contain extracellular matrix proteins, including laminin, collagen-I, and collagen-IV. Further, the resulting acellular tissue can be delivered to a subject in need thereof to facilitate the in vivo generation and/or regeneration of cells and tissues within the subject. The resulting acellular, delipidated tissue product can be formulated to be delivered to a subject in need thereof by any suitable method of deliver, including but not limited to, implantation or injection. The acellular, delipidated tissue products can be enhanced by the addition of one or more other agents or compounds to the acellular, delipidated tissue product, including without limitation other cells (e.g., stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. In some embodiments, the acellular tissue products can be co-administered with one or more other agents or compounds, including without limitation other cells (e.g., stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

In some embodiments, any of the acellular, delipidated tissue scaffolds described herein can be administered to a subject in need thereof in the original form, as an injectable formulation, or as other derivatives of the acellular, delipidated tissue. In some embodiments, the subject in need thereof has a peripheral nerve injury, spinal cord injury, spinal cord disease, amputation or otherwise suffers from degradation of a component of the spine (e.g., intervertebral disc degeneration). In other embodiments, the subject could have osteoarthritis of the hip or knee, trauma to the lungs, volumetric muscle loss, cirrhosis of the liver, or otherwise requires partial or total organ replacement.

### Decellularized, Delipidated Tissue Hydrogels

As previously discussed, the decellularized, delipidated tissues produced from the apoptosis and/or chemical based decellularization and delipidate methods described herein can undergo thermal gelation to form a decellularized tissue hydrogel. FIG. 1D shows a flow diagram outlining the general steps in a method of hydrogel formation using thermal gelation 3000. Decellularized tissue can be obtained from the chemical or apoptotic decellularization methods (1000, 2000) described herein and delipidated in 3010 as described above. In some embodiments, the decellularized tissue is lyophilized. The amount of decellularized, delipidated tissue can range from 0.1 milligrams to about 20 milligrams or more.

The decellularized, delipidated tissue can be enzymatically digested 3020. Enzymatic digest can be completed by digesting the decellularized tissue in pepsin. In some embodiments, the digestion occurs under constant agitation. Enzymatic digestion 3020 can occur by incubating the decellularized tissue in an enzyme in solution for a period of time that can range from about 1 hour to 24, 36, 48, 64 or 72 hours or more. In some embodiments, the enzyme used in the enzymatic digestion is pepsin. The concentration of pepsin used can be about 1 to about 10 mg/mL. The enzyme can be diluted in a deionized water or hydrochloric acid solution to the about 1 to about 10 mg/mL concentration. When a hydrochloric acid solution is used, the hydrochloric acid solution can be about 0.01 M to about 0.1 M HCl. Enzymatic digestion 3020 can be performed at a temperature ranging from about 25° C. to about 37° C.

After enzymatic digestion, the digested tissue can be neutralized 3030 as needed using an appropriate solution (e.g., a sodium hydroxide solution). During or after neutralization the tissue can be further diluted using PBS to a desired tissue concentration. After neutralization or dilution, the solution can be referred to as a pre-gel solution. In some embodiments, the concentration of decellularized tissue in the pre-gel solution can range from about 0.01 mg to 20 mg.

The pre-gel solution can then be used to form a hydrogel via thermal gelation 3040. Thermal gelation 3040 can be performed by incubating the pre-gel solution at about 37° C. for a period of time that can range from about 15 minutes to about 60 minutes. In some embodiments the incubation period of time can be 15, 20, 25, 30, 35, 40, 45, 50, 55, or about 60 minutes. In some embodiments, the pre-gel solution can be mixed with a cargo molecule or cells that can be delivered with the decellularized hydrogel.

In some embodiments thermal gelation can occur ex vivo or in vitro. In some embodiments, it can be desirable for thermal gelation to occur ex vivo or in vitro to form a hydrogel that can be subsequently implanted into a subject. In some of these embodiments, the pre-gel solution can be put in a mold and then incubated to obtain a desired shape or size hydrogel implant. In other embodiments, it can be desirable to deliver the pre-gel solution to a subject and allow the pre-gel solution to thermally gel after delivery to the subject. In some embodiments, this can be accomplished by immediately delivering the pre-gel solution to a subject before the gelation process can take place. In other embodiments, the pre-gel solution can be kept at a temperature lower than the gelation temperature of the pre-gel solution that can delay or prevent hydrogel formation until delivery. Once delivered to the subject, a hydrogel can form inside the subject because the pre-gel solution will be exposed to a temperature of about 37° C. inside the subject.

### Uses of the Decellularized, Delipidated Tissues and Hydrogels Thereof

The decellularized tissues (acellular tissue), pre-gel solution, or hydrogels thereof described herein can be used as a tissue scaffold and hydrogels for the repair of various soft tissue injuries. The resulting acellular, delipidated tissue or hydrogel thereof can be used as a scaffold substrate for three-dimensional (3D) cell culture and tissue engineering techniques. The acellular, delipidated tissue, pre-gel solution, or hydrogels thereof described herein can contain extracellular matrix proteins, including laminin and collagen.

Further, the resulting acellular, delipidated tissue pre-gel solution, or hydrogels thereof described herein can be delivered to a subject in need thereof to facilitate the in vivo generation and/or regeneration of cells and tissues within the subject. The resulting acellular, delipidated tissue product, pre-gel solution, or hydrogel thereof can be formulated to be delivered to a subject in need thereof by any suitable method of deliver, including but not limited to, implantation or injection.

The acellular, delipidated tissue product, pre-gel solution, or hydrogels thereof described herein can be enhanced by the addition of one or more other agents or compounds to the acellular, delipidated tissue product, including without limitation other cells (e.g,. stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. In some embodiments, the acellular, delipidated tissue products, pre-gel solution, or hydrogels thereof described herein can be co-administered with one or more other agents or compounds, including without limitation other cells (e.g., stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. For instance, in embodiments, the acellular, delipidated tissue product, pre-gel solution, or hydrogels thereof described herein can be co-administered with components to aid in cell growth (e.g., growth of cells co-administered and/or in-growth of cells in situ). Such components can include, but are not limited to, nutrients, growth factors, signaling peptides, hormones, cell-secreted biomolecules (such as, but not limited to exosomes), and the like. Also, the disclosed hydrogel can be incorporated with some nanoparticles/microgel loaded with drugs, nerve growth factor.

In some embodiments, any of the acellular, delipidated tissue scaffolds pre-gel solution, or hydrogels thereof described herein can be administered to a subject in need thereof in the original form, as an injectable formulation, or as other derivatives of the acellular, delipidated tissue. In some embodiments, the subject in need thereof has a peripheral nerve injury, spinal cord injury, spinal cord disease, amputation or otherwise suffers from degradation of a component of the spine (e.g., intervertebral disc degeneration). In other embodiments, the subject could have osteoarthritis of the hip or knee, trauma to the lungs, volumetric muscle loss, cirrhosis of the liver, or otherwise requires partial or total organ replacement.

Additional details regarding the methods, compositions, and articles of the present disclosure are provided in the Example below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### Aspects

The present disclosure can be described in accordance with the following numbered aspects, which should not be confused with the claims.

Aspect 1. A tissue hydrogel comprising:
decellularized, delipidated tissue comprising native extracellular matrix proteins,
wherein the decellularized, delipidated tissue is cross-linked to form the hydrogel.

Aspect 2. The tissue hydrogel of aspect 1, further comprising a therapeutic agent.

Aspect 3. The tissue hydrogel of aspect 2, wherein the therapeutic agent is a population of cells, a polynucleotide, an amino acid, a peptide, a polypeptide, an antibody, an aptamer, a ribozyme, a guide sequences for a ribozyme that inhibit translation or transcription of essential tumor proteins and genes, a hormone, an immunomodulator, an antipyretic, an anxiolytic, an antipsychotic, an analgesic, an antispasmodic, an anti-inflammatory agent, an anti-histamine, an anti-infective, a chemotherapeutic, or a combination thereof.

Aspect 4. The tissue hydrogel of any one of aspects 1-3, wherein the decellularized, delipidated tissue is decellularized brain, spinal cord, nerve, heart, lung, liver, muscle, cartilage, tendons, ligament, menisci, stomach, intestine, pancreas, kidney, blood vessel, bone, or cornea tissue.

Aspect 5. The tissue hydrogel of any one of aspects 1-4, wherein the decellularized, delipidated tissue is nervous tissue from a mammal selected from the group consisting of: pig, bovine, and human.

Aspect 6. The tissue hydrogel of any one of aspects 1-5, wherein the decellularized, delipidated tissue hydrogel is formed by the method comprising:
(a) removing cells and DNA from a tissue sample from a donor with a decellularization process that retains native extracellular matrix (ECM) components to produce a decellularized tissue;
(b) removing lipids from the decellularized tissue from (a) with a delipidation process that retains the native ECM components to produce a decellularized, delipidated tissue;
(c) enzymatically digesting the decellularized, delipidated tissue from (b); and
(d) neutralizing the digested, decellularized, delipidated tissue from (c) to form a pre-gel solution; and
(e) exposing the pre-gel solution to temperatures of about 37° C. for an amount of time to form the tissue hydrogel.

Aspect 7. The tissue hydrogel of aspect 6, wherein the decellularization process comprises:
(i) inducing widespread apoptosis in the tissue sample;
(ii) removing apoptotic bodies using a mild hyper or hypo tonic buffered solution; and
(iii) removing DNA by exposing the tissue from (ii) to DNase.

Aspect 8. The tissue hydrogel of aspect 6 or 7, wherein the decellularization process comprises:
(i) incubating the tissue sample in a solution containing sodium deoxycholate or Triton X-100;
(ii) removing DNA from the tissue from (i) by exposing the tissue from (i) to DNase; and
(iii) washing the tissue from (ii) in PBS.

Aspect 9. The tissue hydrogel of aspect 8, wherein the solution in step (i) also includes SB-10, SB-16, or a combination thereof.

Aspect 10. The tissue hydrogel of any of aspects 6-9, wherein the delipidation process comprises:
(i) incubating the decellularized tissue from (a) with a delipidation solvent solution for an amount of time to remove lipids; and
(ii) washing the tissue from (i) to remove residual lipids.

Aspect 11. The tissue hydrogel of aspect 10, wherein the delipidation solvent is selected from the group consisting of: a combination of ethanol and dichloromethane; a combination of chloroform and methanol; a combination of hexane and isopropanol; and type I water.

Aspect 12. The tissue hydrogel of aspect 11, wherein the decellularized, delipidated tissue is porcine nervous tissue, wherein the tissue sample is incubated in a solution containing sodium deoxycholate, and wherein the delipidation solvent is a combination of ethanol and dichloromethane.

Aspect 13. The tissue hydrogel of any one of aspects 6-12, wherein the delipidation process is repeated a second time.

Aspect 14. The tissue hydrogel of aspect 13, wherein the delipidation solvent for the second delipidation process is the same as the delipidation solvent in step (i).

Aspect 15. The tissue hydrogel of aspect 13 or 14, wherein the delipidation solvent comprises a combination of ethanol and dichloromethane in a ratio of about 1:1 to about 1:4.

Aspect 16. The tissue hydrogel of aspect 15, wherein the ratio of ethanol to dichloromethane is about 1:2.

Aspect 17. The tissue hydrogel of aspect 11, wherein the ratio of hexane to isopropanol is about 3:2.

Aspect 18. The tissue hydrogel of any of aspects 6-17, wherein step (e) is performed in vivo.

Aspect 19. The tissue hydrogel of any of aspects 6-18, wherein step (c) is performed by incubating the tissue from (b) in a solution comprising pepsin.

Aspect 20. The tissue hydrogel of any of aspects 6-19, further comprising the step of lyophilizing the tissue from step (b) before step (c).

Aspect 21. The tissue hydrogel of any of aspects 6-20, further comprising adding a crosslinking agent to the tissue from step (d) before step (e) to reduce gelation time.

Aspect 22. The tissue hydrogel of any one of aspects 6-21, wherein the method to form the tissue hydrogel additionally comprises (i) washing the tissue hydrogel with water to remove bound salts and buffers.

Aspect 23. The tissue hydrogel of aspect 22, wherein washing with water is conducted for from about 24 to about 48 hours.

Aspect 24. The tissue hydrogel of aspect 22 or 23, wherein washing with water is performed after step (b) and before step (c).

Aspect 25. The tissue hydrogel of aspect 24, wherein the decellularized, delipidated tissue is human nervous tissue, wherein the tissue sample is incubated in a solution containing Triton X-100, wherein the delipidation solvent is a combination of ethanol and dichloromethane, and wherein the delipidation solvent for the second delipidation process is a combination of ethanol and dichloromethane.

Aspect 26. A method comprising:
administering a tissue hydrogel of any one of aspects 1-25 to a subject.

Aspect 27. The method of aspect 26, further comprising co-administering a population of cells with the tissue hydrogel.

Aspect 28. The method of aspect 26 or 27, wherein the subject has a peripheral nerve injury, spinal cord injury, spinal cord disease, amputation, has degradation of a component of the spine, osteoarthritis, lung trauma, volumetric muscle loss, or liver cirrhosis.

Aspect 29. A pre-gel solution comprising:
a solution of digested, decellularized, delipidated tissue comprising native extracellular matrix proteins,
wherein the solution of digested, decellularized, delipidated tissue is capable of gelation to form a hydrogel at about 37° C.

Aspect 30. The pre-gel solution of aspect 29, wherein the pre-gel solution is liquid at about 4° C. to about 25° C.

Aspect 31. The pre-gel solution of aspect 29 or 30, further comprising a therapeutic agent.

Aspect 32. The pre-gel solution of aspect 31, wherein the therapeutic agent is a population of cells, a polynucleotide, an amino acid, a peptide, a polypeptide, an antibody, an aptamer, a ribozyme, a guide sequences for a ribozyme that inhibit translation or transcription of essential tumor proteins and genes, a hormone, an immunomodulator, an antipyretic, an anxiolytic, an antipsychotic, an analgesic, an antispasmodic, an anti-inflammatory agent, an anti-histamine, an anti-infective, a chemotherapeutic, a growth medium, a growth factor, an exosome, or a combination thereof.

Aspect 33. The pre-gel solution of any one of aspects 29-32, further comprising a crosslinking agent.

Aspect 34. The pre-gel solution of any one of aspects 29-33, wherein the digested, decellularized, delipidated tissue is brain, spinal cord, nerve, heart, lung, liver, muscle, cartilage, tendons, ligament, menisci, stomach, intestine, pancreas, kidney, blood vessel, bone, or cornea tissue.

Aspect 35. The pre-gel solution of any one of aspects 29-34, wherein the digested, decellularized, delipidated tissue is porcine nervous tissue or human nervous tissue.

Aspect 36. The pre-gel solution of any one of aspects 29-35, wherein the pre-gel solution is formed by the method comprising:
(a) removing cells and DNA from a tissue sample from a donor with a decellularization process that retains native extracellular matrix (ECM) components to produce a decellularized tissue;
(b) removing lipids from the decellularized tissue from (a) with a delipidation process that retains the native ECM components to produce a decellularized, delipidated tissue;
(c) enzymatically digesting the decellularized, delipidated tissue from (b); and
(d) neutralizing the digested, decellularized, delipidated tissue from (c) to form the pre-gel solution.

Aspect 37. The pre-gel solution of aspect 36, wherein the decellularization process comprises:
(i) inducing widespread apoptosis in the tissue sample;
(ii) removing apoptotic bodies using a mild hyper or hypo tonic buffered solution; and
(iii) removing DNA by exposing the tissue from (ii) to DNase.

Aspect 38. The pre-gel solution of aspect 36 or 37, wherein the decellularization process comprises:
(i) incubating the tissue sample in a solution containing sodium deoxycholate or Triton X-100;
(ii) removing DNA from the tissue from (i) by exposing the tissue from (i) to DNase; and
(iii) washing the tissue from (ii) in PBS.

Aspect 39. The pre-gel solution of aspect 38, wherein the solution in step (i) also includes SB-10, SB-16, or a combination thereof.

Aspect 40. The pre-gel solution of any one of aspects 36-39, wherein the delipidation process comprises:
(i) incubating the decellularized tissue from (a) with a delipidation solvent solution for an amount of time to remove lipids; and
(ii) washing the tissue from (i) to remove residual lipids.

Aspect 41. The pre-gel solution of aspect 40, wherein the delipidation solvent is selected from the group consisting of: a combination of ethanol and dichloromethane; a combination of chloroform and methanol; a combination of hexane and isopropanol; and type I water.

Aspect 42. The pre-gel solution of aspect 41, wherein the decellularized, delipidated tissue is porcine nervous tissue, wherein the tissue sample is incubated in a solution containing sodium deoxycholate, and wherein the delipidation solvent is a combination of ethanol and dichloromethane.

Aspect 43. The pre-gel solution of aspect 40 or 41, wherein the delipidation process is repeated a second time.

Aspect 44. The pre-gel solution of aspect 43, wherein the delipidation solvent for the second delipidation process is the same as the delipidation solvent in step (i).

Aspect 45. The pre-gel solution of any one of aspects 40-44, wherein the delipidation solvent comprises a combination of ethanol and dichloromethane in a ratio of about 1:1 to about 1:4.

Aspect 46. The pre-gel solution of aspect 45, wherein the ratio of ethanol to dichloromethane is about 1:2.

Aspect 47. The pre-gel solution of aspect 41, wherein the ratio of hexane to isopropanol is about 3:2.

Aspect 48. The pre-gel solution of any one of aspects 36-47, wherein the method to form the pre-gel solution additionally comprises (i) washing the tissue hydrogel with water to remove bound salts and buffers.

Aspect 49. The pre-gel solution of aspect 48, wherein washing with water is conducted for from about 24 to about 48 hours.

Aspect 50. The pre-gel solution of aspect 48 or 49, wherein washing with water is performed after step (b) and before step (c).

Aspect 51. The pre-gel solution of aspect 50, wherein the decellularized, delipidated tissue is human nervous tissue, wherein the tissue sample is incubated in a solution containing Triton X-100, wherein the delipidation solvent is a combination of ethanol and dichloromethane, and wherein the delipidation solvent for the second delipidation process is a combination of ethanol and dichloromethane.

Aspect 52. The pre-gel solution of any one of aspects 36-51, wherein step (c) is performed by incubating the tissue from (d) in a solution comprising pepsin.

Aspect 53. The pre-gel solution of any one of aspects 36-52, further comprising lyophilizing the tissue from step (b) before step (c).

Aspect 54. A method comprising:
injecting an amount of a pre-gel solution of aspect 29 into a subject.

Aspect 55. The method of aspect 54, comprising: co-administering a population of cells with the pre-gel solution.

Aspect 56. The method of aspect 54 or 55, wherein the subject has a peripheral nerve injury, spinal cord injury, spinal cord disease, amputation, has degradation of a component of the spine, osteoarthritis, lung trauma, volumetric muscle loss, or liver cirrhosis.

Aspect 57. A method of making a pre-gel solution, the method comprising:
(a) removing cells and DNA from a tissue sample from a donor with a decellularization process that retains native extracellular matrix (ECM) components to produce a decellularized tissue;
(b) removing lipids from the decellularized tissue from (a) with a delipidation process that retains the native ECM components to produce a decellularized, delipidated tissue;
(c) enzymatically digesting the decellularized, delipidated tissue from (b); and
(d) neutralizing the digested, decellularized, delipidated tissue from (c) to form the pre-gel solution.

Aspect 58. The method of aspect 58, wherein the decellularization process comprises:
(i) inducing widespread apoptosis in the tissue sample;
(ii) removing apoptotic bodies using a mild hyper or hypo tonic buffered solution; and
(iii) removing DNA by exposing the tissue from (ii) to DNase.

Aspect 59. The method of aspect 58 or 59, wherein the decellularization process comprises:
(i) incubating the tissue sample in a solution containing sodium deoxycholate or Triton X-100;
(ii) removing DNA from the tissue from (i) by exposing the tissue from (i) to DNase; and
(iii) washing the tissue from (ii) in PBS.

Aspect 60. The method of aspect 59, wherein the solution in step (i) also includes SB-10, SB-16, or a combination thereof.

Aspect 61. The method of any one of aspects 58-60, wherein the delipidation process comprises:
(i) incubating the decellularized tissue from (a) with a delipidation solvent solution for an amount of time to remove lipids; and
(ii) washing the tissue from (i) to remove residual lipids.

Aspect 62. The method of aspect 61, wherein the delipidation solvent comprises a combination of ethanol and dichloromethane in a ratio of about 1:1 to about 1:4.

Aspect 63. The method of any one of aspects 58-62, wherein step (c) is performed by incubating the tissue from (d) in a solution comprising pepsin.

Aspect 64. The method of any one of aspects 58-63, further comprising the step of lyophilizing the tissue from step (b) before step (c).

Aspect 65. The method of any one of aspects 58-64, further comprising adding a cross-linking agent to the pre-gel solution.

Aspect 66. A method of making a decellularized, delipidated tissue hydrogel, the method comprising:
preparing a pre-gel solution according to the method of any one of aspects 58-65; and
exposing the pre-gel solution to temperatures of about 37° C. for an amount of time to form the decellularized, delipidated tissue hydrogel.

Aspect 67. The method of aspect 66, wherein the pre-gel solution is injected into a patient and the hydrogel is formed in vivo.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein.

### Example 1: Decellularized Porcine Sciatic Nerve Based Hydrogel for Tissue Regeneration

Introduction: Xenogeneic decellularized matrices or hydrogels derived from decellularized tissues or organs are highly attractive scaffolds for surgical applications due to their low immunogenicity, biocompatibility, tunability, and tissue specific extracellular matrix (ECM) components. Previously, porcine decellularized nerve matrix hydrogel have shown potential in peripheral nerve regeneration (Lin 2018). However, such hydrogels were not able to be formed as a hydrogel in vivo and had to be created ex vivo. The present example explores the decellularization of different branches of porcine sciatic nerves (peroneal, tibial, sural and sciatic) and fabrication of such nerves into injectable hydrogels. Decellularization efficiency, gelation kinetics and mechanical properties of resulting hydrogels were assessed, and the effect of lipid presence on hydrogel fabrication and properties. These experiments were conducted to generate an injectable, decellularized peripheral nerve hydrogel with ECM protein composition and mechanical properties that mimic native nerves, which can be used for neural regeneration after injury.

### Methods:

Porcine peripheral nerves with different branches (peroneal, sural, tibial and sciatic nerve) were isolated from mini pigs, and decellularized using a previously published protocol for rodent sciatic nerve (McCrary et al 2020, incorporated by reference herein, above) using a novel sodium deoxycholate-based chemical decellularization method for peripheral nerve. Briefly, nerves were subjected to 7 hours of washes in water, followed by 18 hours of washes in 125 mM sulfobetaine (SB-10) buffer. Thereafter, the nerves were washed with 100 mM Na/10 mM Phosphorus buffer for 15 minutes, and then with 3% SD/SB-16 solution for 1.5 hours, with 50 mM Na/10 mM phosphorus buffer thrice, for 15 minutes each. To remove DNA content, the nerves were subjected to 75 U/mL of DNAse solution for 3 hours. Followed by the washing with 50 mM Na/10 mM phosphorus buffer thrice, for 1 hour each. The nerves were then washed once with 0.2 U/mL of chondroitinase ABC enzyme solution. Finally, the decellularized nerves were washed with 1×PBS for 3 hours.

Decellularization efficiency was assessed using immunofluorescence staining for removal of cellular components (DAPI-nuclear presence, S100-Schwann cells, MBP-myelin, and β-tubulin III-neurons) and preservation of ECM proteins (collagen-I, collagen-IV, and laminin). Fresh peripheral nerve branches were used as controls. Decellularization efficiency was assessed using immunofluorescence staining on cryosectioned decellularized nerves for removal of cellular components (DAPI-nuclear presence, S100-Schwann cells, MBP-myelin, and β-tubulin III-neurons) and preservation of ECM proteins (collagen-I, collagen-IV, and laminin).

For delipidation experiments, after decellularization, the porcine nerves were delipidated (also referred to herein as “delipidized”). In early trials some solvents known in the literature to remove lipids from tissue were used, but the remaining tissue after delipidation was not able to undergo gelation kinetics. Solvents such as PBS, hexane, acetone, choloform, isopropyl alcohol were studied to remove lipids from the decellularized nerve, but while these solvents remove lipid residuals efficiently, when they were tested, the decellularized and delipidated nerves were not able to undergo gelation kinetics. Thus, other delipidation solvents were tried, including using a solution of ethanol and dichloromethane in a 1:2 ratio for 24 hours. This solution was used to remove residual lipids and then washed with distilled water thrice, 30 minutes each. The lipid content was then measured using the sulfo-phospho-vanillin reaction. Non-delipidized decellularized nerves were used as controls.

Both decellularized only and decellularized and delipidated nerves were lyophilized and stored at −80° C. until further use. Decellularized nerves (delipidized and non-delipidized) were then digested at a concentration of 10 mg/mL using pepsin/acid solution and then neutralized to yield hydrogel pre-gel solutions. The gelation kinetics of the pre-gel solutions were assessed using U.V. spectrophotometer at 37° C. The mechanical strength of the resulting hydrogels was measured using rheology (parallel plate geometry).

To assess the gelation kinetics, decellularized nerves were then digested at a concentration of 13.4 mg/mL using 1 mg/mL of pepsin/acid solution for 72 hours, and then neutralized with 0.1 N NaOH, and 10×PBS, to yield hydrogel pre-gel solutions (of 10 mg/mL final concentration). The gelation kinetics of the pre-gel solutions were assessed by measuring absorbance at 405 nm every 30s for up to 30 minutes, using U.V. spectrophotometer at 37° C. The absorbance data were then normalized using previously published protocol. The mechanical strength of the resulting hydrogels was measured using Anton Parr MCR 302 rheometers thermal gelation app with 25 mm parallel plate geometry.

Earlier attempts were made to fabricate injectable hydrogels using decellularized lipids, however, it was found that the presence of lipids even after decellularization of nerves, was interfering in hydrogel formation. Hence, it was attempted to remove lipids, by treating decellularized nerves using ethanol:dichloromethane (1:2) for 24 hours, at room temperature, as per previous literature. After delipidating the nerves, the decellularized nerves were then digested at concentration 13.4 mg/mL, using 1 mg/mL pepsin solution for 72 hours at room temperature, followed by, neutralization to pH 7.4, using 0.1 N NaOH and then adding 1/9^{th} volume of 10×PBS. The final concentration of pre-gel solution was 10 mg/mL. this solution was used to analyze the gelation kinetics using U.V. spectrophotometer. Rheological thermal gelation study was performed on 10 mg/mL pre-gel solution using Anton Par MCR 302 instrument. For rheological analysis, neutralized pre-gel solution was kept at 4° C. and then temperature was increased to 37° C., and storage and loss modulus was measured at 1% strain and 1% Hz frequency. Thereafter, the instrument was allowed to maintain temperature at 37° C. for next 15 mins, and storage and loss modulus value was measured.

### Results:

Analysis of decellularization efficiency: As the SDD method of decellularization had never been implemented on porcine nerve, it was first desired to quickly assess the extent of decellularization this process causes on larger nerves and determine if this process would need to be altered to accommodate these nerves in the future.

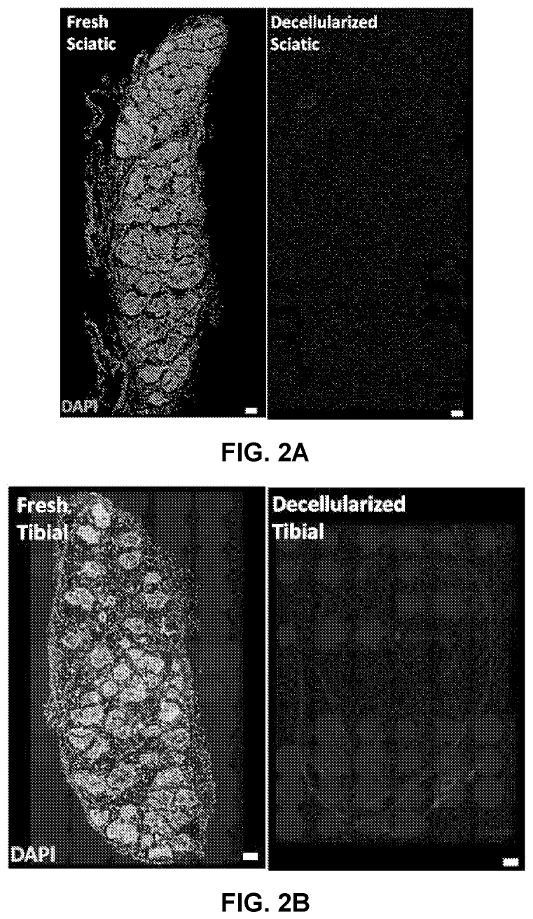

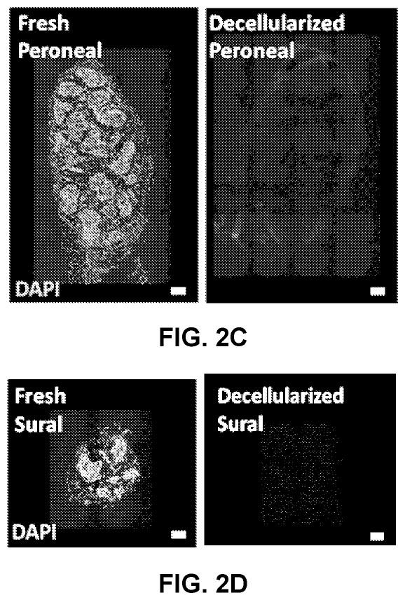

First, cellular removal was assessed using immunohistochemistry for key intracellular components. An example of this removal is demonstrated in FIGS. 2A-2D showing nuclei through DAPI staining on fresh porcine nerves and SDD decellularized porcine nerves. DAPI staining was used to assess nuclear presence after decellularization. Decellularization resulted in near complete removal of nuclei as evidenced by no visible DAPI staining compared to fresh nerve controls. Overall, there was clear removal of cellular debris, shown in dramatic reduction in nuclear staining, regardless of nerve diameter (FIGS. 2A-2D).

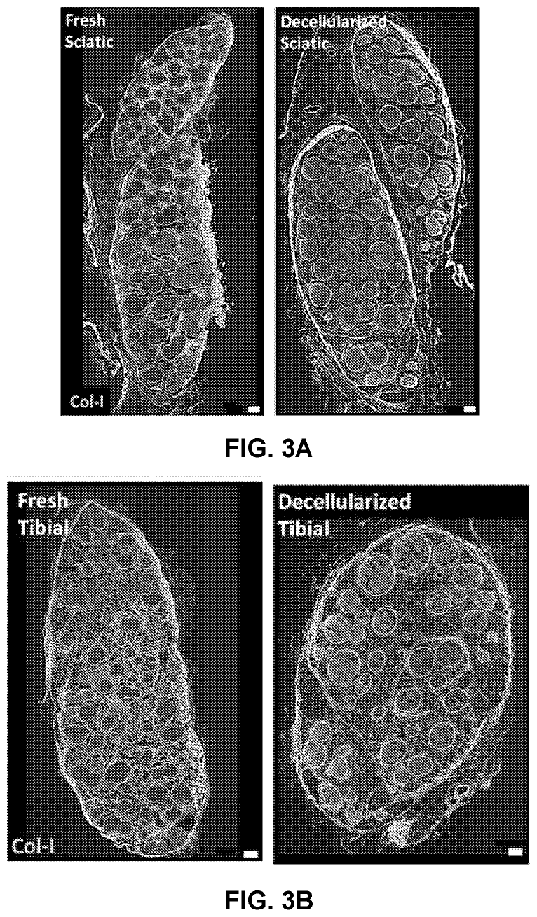

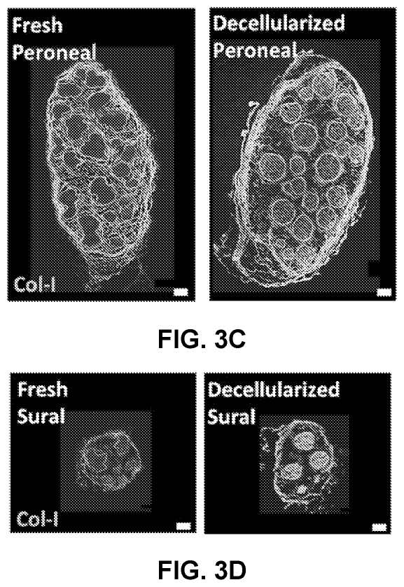

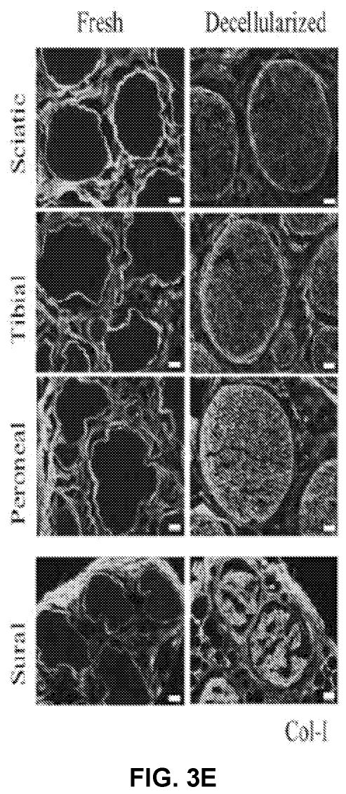

The Effect of Decellularization on ECM preservation: After confirming for DAPI absence in decellularized nerves, the presence of Collagen-I (Col-1) content and structure were also investigated in decellularized porcine sciatic nerve. The main aim for this experiment was to analyze the effect of decellularization on extracellular matrix (ECM) preservation. Cross sectional images staining for Collagen-I in fresh and decellularized sciatic, tibial, peroneal, and sural nerve are shown in FIGS. 3A-3E. Scale bars=50 μm.

Collagen-I, Collagen IV and laminin are key ECM components in peripheral nerve and should be preserved during decellularization as possible. Staining for Collagen-I, Collagen IV and laminin demonstrated robust preservation of these ECM proteins (Col-I shown in FIGS. 3A-3E). These results demonstrated that the decellularization method employed was effective at removing cellular components and retaining the native ECM. These results suggest that the SDD method of decellularization can be used on larger mammal nerves with little to no optimization.

Gelation kinetics of the decellularized, nerve-based hydrogels was conducted, and results demonstrated that digested, decellularized, porcine sciatic nerve branches can successfully form hydrogels. Gelation time varied based on sciatic nerve branch. However, the resulting hydrogels from all nerve branches lacked sufficient mechanical strength. From these results, it was believed that the presence of lipids in the decellularized porcine nerves impeded gelation, thereby negatively impacting mechanical stability.

Delipidation and Lipid Estimation in Decellularized Peripheral Nerves: After decellularization, a part of the nerve was delipidated using ethanol:dichloromethane (1:2) for 24 hours at RT and washed with water thrice for 30 min each, while remaining half was used as a control to assess the effect of lipid presence on gelation kinetics. The presence of lipids was quantified using sulfo-phopsho vanillin reagent. As demonstrated in FIGS. 4A-4D, decellularized nerves that were delipidated had a significantly lower amount of lipids as compared to the nerves that were only decellularized. Data were analyzed from three different pigs, statistical analysis: t test unpaired, *p≤0.05, **p≤0.01, ***p≤0.001. The presence of lipids in decellularized peripheral nerves varied in different branches. Hence, upon removal of lipids using ethanol and dichloromethane solvent, it was observed that the decellularized, delipidated nerve had varied residual lipids remaining. For instance, sciatic decellularized nerves had lipid concentration of approximately 1300 mg/dL, while delipidated, decellularized sciatic nerves had lipid concentration approximately 450 mg/dL.

Gelation Kinetics Analysis using UV spectrophotometer: After the nerves were decellularized and delipidate (decellularized-only nerves were also used as control), the nerves were digested using 1 mg/mL pepsin solution at nerve concentration of 13.4 mg/mL for 72 hours at room temperature, and then neutralized using 0.1N NaOH solution and then 1/9^{th} Volume of 10×PBS was added. To make up to final 10 mg/mL concentration for ECM, 1×PBS was used for dilution. The solutions were kept at 4° C. throughout, and then kept in UV absorbance plate reader pre-set at 37° C. The absorbance was read for 30 min every 30 seconds, at 405 nm. The absorbance value obtained was normalized as per previously published literature and normalized absorbance versus time was plotted. The time taken for 50% gelation (when normalized absorbance reaches near 0.5, and time taken for 95% gelation (when normalized absorbance reaches near 0.95) was analyzed. For instance, decellularized and delipidized sural nerve pre-gel reached 95% gelation 1.5 times faster than that with higher lipids. Overall, it was seen that the presence of lipids delayed hydrogel gelation.

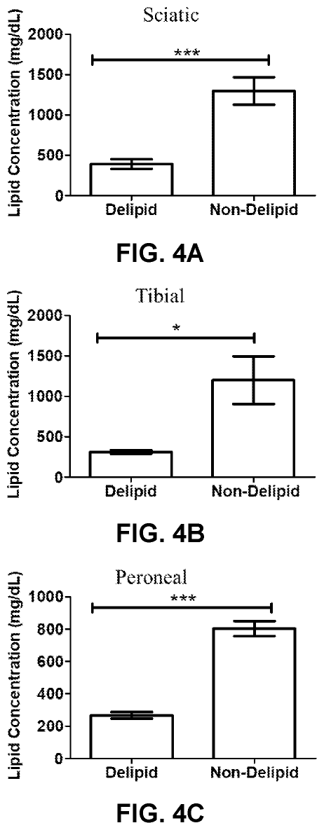

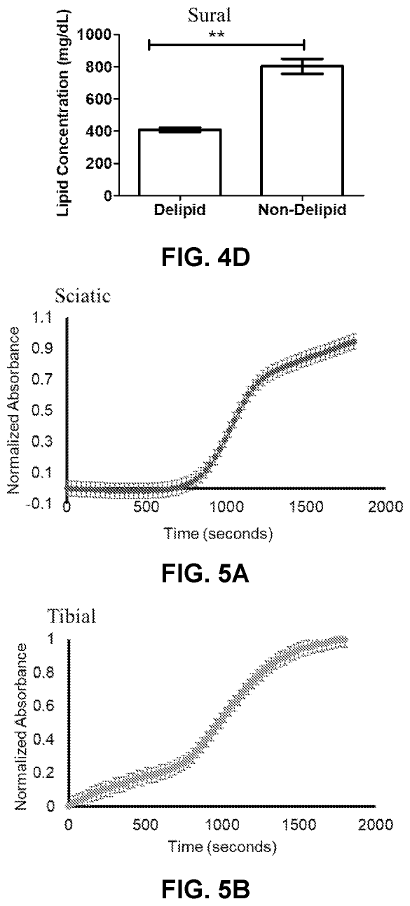

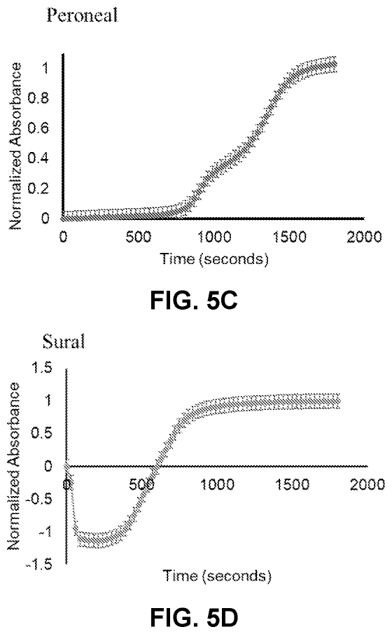

As shown in FIGS. 5A-5B, decellularized and delipidated nerves were able to gel at 37° C., while decellularized-only nerves did not undergo gelation with time. This shows that lipid removal resulted in better and faster gelation kinetics. Time taken for 50% and 95% gelation is shown in Table 1. Sural decellularized and delipidated nerves were shown to have a faster gelation kinetics as compared to other peripheral nerves branches. Since some decellularized, delipidated nerves are still taking a little longer for gelation than desired for certain clinical applications, addition of crosslinking agents to accelerate gelation at body temperature is also being evaluated. Such crosslinkers can be added at the pre-gel stage, as crosslinking the pre-gel solution can further accelerate the gelation kinetics.

TABLE 1
Gelation Kinetics

| Nerve | T50 (min) | T95 (min) |
|---|---|---|
| Sciatic | 18 | 29.5 |
| Tibial | 16.5 | 26 |
| Peroneal | 21 | 25.5 |
| Sural | 12 | 19 |

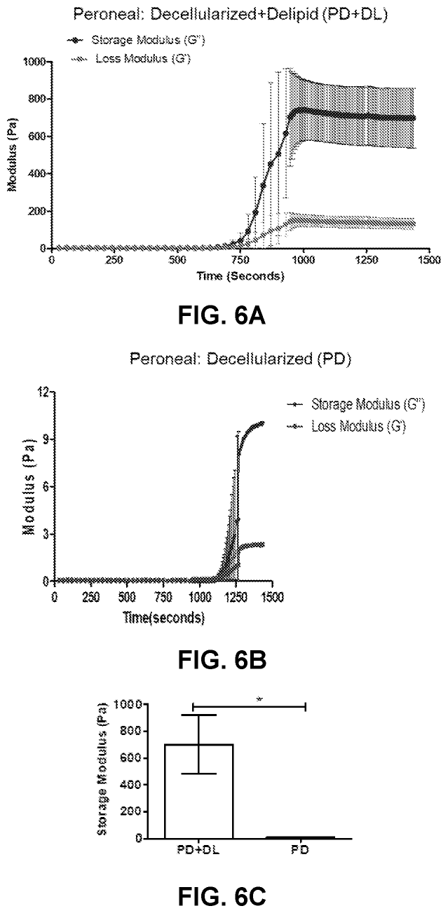

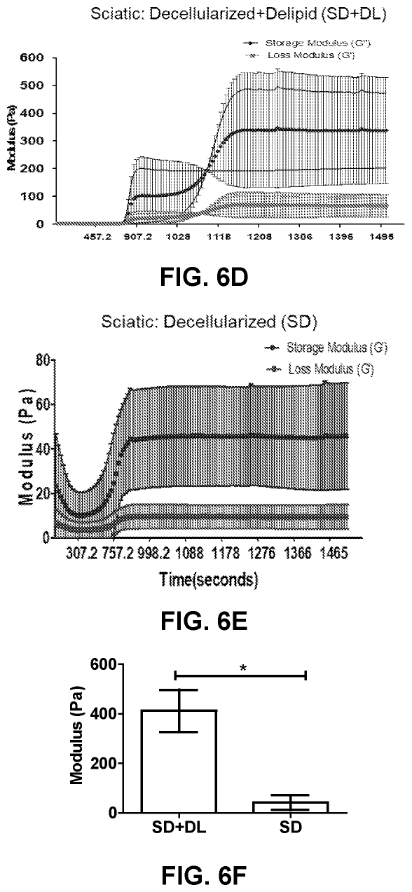

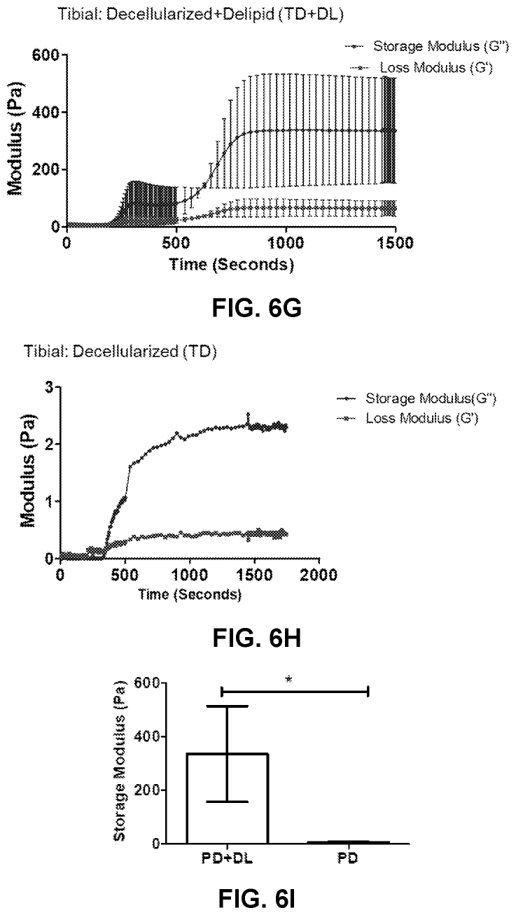

Thermal Gelation kinetics of decellularized and delipidated peripheral nerves pre-gel solutions: Next mechanical strength of the tissues was assessed via rheology and results are presented in FIGS. 6A-6F. After the nerves were decellularized and delipidated (decellularized-only nerves were also used as control), the nerves were digested using 1 mg/mL pepsin solution at nerve concentration of 13.4 mg/mL for 72 hours at room temperature, and then neutralized using 0.1N NaOH solution and then 1/9th Volume of 10×PBS was added. To make up to final 10 mg/mL concentration for ECM, 1×PBS was used for dilution. The solutions were kept at 4° C. throughout, and then loaded on Anton Par MCR 302 rheometer instruments. The parallel plate geometry was used for measurement, and thermal gelation kinetics was studied from 4° C. to 37° C., and then instrument was kept at 37° C. for next 30 min, to allow the pre-gel solutions to form hydrogel. The storage and loss modulus value were analyzed, and the storage modulus at the end of 30 min incubation time was reported. As shown in FIGS. 6A-6F, the decellularized and delipidated nerves had higher storage modulus value as compared to only decellularized nerves, indicating that lipid removal enabled efficient gelation of hydrogel, resulting in mechanical strength of hydrogel sufficient for neural tissue regeneration.

### Conclusion:

This example explored the decellularization of different porcine sciatic nerve branches and subsequent fabrication into injectable hydrogels to be used for neural regeneration after injury. The ability to robustly remove the cellular components while preserving the native ECM proteins was demonstrated. The ability to fabricate injectable hydrogels from these decellularized porcine nerve branches was also demonstrated. It was found that the presence of lipids delays gelation kinetics and ultimately affects the mechanical strength of hydrogel. Removal of the lipid content resulted in improved gelation kinetics and mechanical stability. Overall, injectable hydrogels based on decellularized porcine sciatic nerve branches are a promising scaffold for neural regeneration after injury.

### Example 2: Additional Studies of Porcine Peripheral Nerve Decellularization

### Methods:

Decellularization of porcine peripheral nerves: Peripheral nerve branches (peroneal and sciatic) were isolated aseptically from mini pigs, supplied by the University of Florida (UF) animal care facilities. Nerves were not isolated from live pigs; post-mortem animals were used for nerve isolation. After isolation, the nerves were kept at −30° C. until further use. Decellularization of peripheral nerves was adapted from methods previously used for rodent sciatic nerves. All the chemicals were of standard quality unless specified. Briefly, nerves were subjected to 7 hours of washing in water, followed by 18 hours of washing in 125 mM sulfobetaine (SB-10) (Sigma Aldrich, D4266) buffer. Thereafter, the nerves were washed with 100 mM sodium/10 mM phosphate buffer for 15 minutes, and then with 3% sodium deoxycholate (SD) (Sigma Aldrich, D6750)/SB-16 (Sigma Aldrich, H6883) solution for 1.5 hours, with 50 mM Na/10 mM phosphate buffer thrice, for 15 minutes each. To remove DNA content, the nerves were subjected to 75 U/mL of DNAase solution (Sigma Aldrich, D4527) for 3 hours. Subsequently, the nerves were washed with 50 mM Na/10 mM phosphate buffer thrice, for 1 hour each. The nerves were then washed once with 0.2 U/mL of Chondroitinase ABC enzyme (Sigma Aldrich, C3667) solution. Finally, the nerves were washed with 1×PBS for 3 hours and stored at −30° C. until further use. To understand the effect of remaining lipids after decellularization on injectable hydrogel formulation and gelation kinetics, a part of decellularized nerve piece was subjected to ethanol (Fischer Scientific, NC1150367) and dichloromethane (Sigma Aldrich, 270997-2L) in a 1:2 ratio for 24 hours to remove residual lipids, and then washed with distilled water thrice, 30 minutes each. The decellularized and delipidated nerves were then lyophilized and stored at −80° C. until further use. These nerves are referred to as “decell+delipid” in the rest of the manuscript. Decellularized nerves without delipidation were used as controls and referred to as “decell only” herein.

Immunohistochemistry analysis: For immunohistochemistry (IHC), after decellularization, nerves were fixed using 4% paraformaldehyde and stored in 30% sucrose for 1 week. Nerves were then trimmed, soaked in optimal cutting temperature (OCT) compound at 4° C. overnight, and flash frozen at −80° C. for at least 5 min. OCT blocks were then stored at −20° C. until sectioning. Frozen sections were obtained using a Leica CM1950 cryostat (Leica Biosystems, Germany) with a tissue thickness of 10 μm. Sections were stained using antibodies against collagen-I (Col-1) (Millipore Sigma; C2456, 1:500), collagen-IV (Col-IV) (Abcam; ab6586, 1:500), laminin (Millipore Sigma; L9393, 1:500), β-III tubulin for axons (Abcam; ab7751, 1:1000), S-100 for Schwann cells (Millipore Sigma; S2644, 1:500), myelin basic protein (MBP) for myelin (Millipore Sigma; M3821, 1:150), and 4′,6-diamidino-2-phenylindole (DAPI) for nuclei (Thermo Fisher Scientific; D1306, 1:1000). Negative controls (no primary antibody) were prepared to ensure autofluorescence or nonspecific staining was minimal.

Decellularized Nerve Digestion: Decellularized peripheral nerves were solubilized using the SDD method published previously. Immediately after the delipidation treatment, processed nerves were subject to three 30-min washes in sterile ddH₂O on an orbital rotator, flash-frozen in liquid nitrogen, and then lyophilized (Labconco) for 3 days. Lyophilized nerves were weighed and minced into small pieces in a scintillation vial (Millipore Sigma). Pepsin at 1 mg/mL (Sigma Aldrich P7012) in 0.01 M hydrochloric acid (Sigma Aldrich 258,148) was added to achieve tissue concentrations of 13.4 mg of tissue per 1 mL of pepsin solution. Vials were capped and incubated for 72 hours at room temperature under constant agitation through use of a magnetic stir bar. Nerve digests, which at this point were homogenous solutions, were transferred from scintillation vials into microcentrifuge tubes and placed on ice. Then, 0.1 M NaOH was added to the digests until the pH reached 7.4 to neutralize the solution, and the solution was stabilized by adding 10×PBS at an amount 1/9th the volume of digest. The neutralized solution is referred to as “pre-gel” solution and was used for lipid estimation and rheometry as explained below. Decellularized nerves without delipidation treatment were digested similarly and used as controls.

Lipid estimation in decellularized nerves: To understand the effect of remaining lipids on gelation kinetics of injectable hydrogels, lipid content in decellularized nerves was initially estimated using the Lipid Assay Kit (unsaturated fatty acids) supplied by Abcam (catalogue number: ab242305). The Lipid Assay Kit allows measurement of the lipid content (unsaturated fatty acids only) of samples using sulfo-phospho vanillin reagent by colorimetric absorbance. Briefly, nearly 15 μl of pre-gel solution both decellularized with and without delipidation treatment were placed into a 96-well plate, and then incubated at 90° C. for 30 minutes. The samples were transferred to 4° C. for 5 minutes, following which 150 μL of 18 M sulfuric acid was added to each well. The samples were then incubated at 90° C. for 10 minutes, then at 4° C. for 5 minutes. 100 μL of each sample was then transferred into a clean 96-well plate and background absorbances were read at 540 nm. Following which, 100 μL of sulfo-phospho vanillin reagent was added to each well and mixed well with a pipette. The samples were then incubated at 37° C. for 15 minutes, and absorbance was read at 540 nm. The background absorbance was subtracted from each well. The concentration of lipids in samples were extrapolated from a standard curve plotted using the lipid standard supplied by the manufacturer.

Crosslinking of decellularized nerve pre-gel solution: Pregel solutions were crosslinked individually using different concentrations of genipin (Sigma Aldrich G4796). Different concentrations of genipin (dissolved in DMSO, stock 89 mM) were added to 10 mg/mL neutralized pre-gel solution to achieve final concentrations of 1, 5, 10 mM genipin and incubated at 37° C. to undergo gelation.

Rheological analysis: Frequency sweep and shear thinning experiments were carried out using an Anton Paar MCR 302 in parallel plate geometry with 1 mm gap and 8 mm sandblasted parallel plate. For frequency sweep studies, the hydrogels were cast in 8 mm silicone molds by pouring 125 μL of pre-gel neutralized solution with/without genipin crosslinker. Frequency varied from 0.01 to 100 Hz at constant 1% strain at 25° C., and the storage (G′) and loss (G″) moduli were recorded. Shear thinning experiments were conducted by placing 125 μL of neutralized pre-gel solution with/without crosslinker and shear rate was varied from 0-150 Pa and temperature was constant at 25° C. and viscosity was recorded.

In vitro astrocyte-Schwann cell co-culture analysis: Rat astrocytes (P 2-3) and rat Schwann cells (P 2-4) were cultured in astrocytes/Schwann media provided by Sciencell manufacturer, with addition of 10% fetal bovine serum and 1% penicillin-streptomycin. The cells were cultured in 5% CO₂ at 37° C., and media was changed every 2-3 days. Initially, to determine efficient lipopolysaccharide (LPS) concentration for inflamed astrocytes, different concentrations of LPS (0, 1, 5, and 10 μg/mL) were added to the astrocyte culture media and exposed to ~1×10⁵ rat astrocytes cultured in 6-well plates for 24 hours. Thereafter, the rat astrocytes were fixed with 4% paraformaldehyde (PFA) for 15 minutes and immunostained using glial fibrillar acidic protein (GFAP) marker. The GFAP intensity was measured using Image J software to determine accurate LPS concentration for inflammatory astrocytes. For co-culture experiments, ~0.05×10⁶ rat astrocytes were cultured on top of 24 well culture inserts, while ~0.05×10⁶ rat Schwann cells within 10 mg/mL hydrogel or alone were cultured on bottom of 24-well cell culture plate. 5 μg/mL LPS in 1:1 Schwann: Astrocyte complete media was added to the well and incubated for 48 hours. For rat Schwann cells within hydrogels, 15 mg/mL decellularized peripheral nerves were minced into small pieces using sterilized micro scissors and digested in pepsin solution (1 mg/mL) for 48 hours. After complete digestion, the solution was neutralized using appropriate volume of 0.2 μm syringe filtered 1 N NaOH and 10×PBS. An appropriate volume of rat Schwann cell suspension having ~0.05×10⁶ rat Schwann cells in each hydrogel was added to the neutralized pre-gel solution and a hydrogel concentration of 10 mg/mL was achieved by adding cell culture media. The pre-gel solutions with cells were cast into 8 mm silicone molds and incubated at 37° C., 5% CO₂ for 20 minutes. For genipin-crosslinked hydrogel groups, the required volume of genipin was added to neutralized pre-gel solution containing cell suspension. After 48 hours of incubation, the cells within the hydrogel or alone were stained using live/dead stain as per Thermo Fisher manufacturer recommendations. The hydrogel/cells were then imaged using a Leica 880 confocal, and the live/dead cell ratios were analyzed using Image J software.

Injectability analysis: To determine injectability of the hydrogel solution, 10 mg/mL neutralized pre-gel solution with or without genipin solution was passed through a 25-G ⅝-inch syringe at room temperature in a 96-well plate.

In vitro Schwann cell post-injection biocompatibility analysis: Decell+delipid peroneal and sciatic nerve hydrogels were digested and neutralized to pH 7.4, as previously explained above. Rat Schwann cells (P 4-5, 1×10⁵) were added to neutralized pre-gel solutions and final concentrations of hydrogels were adjusted to 10 mg/mL using Schwann cell culture complete media. 1 mM genipin was added to the hydrogel cell solution and then the solution was passed through a 25-G needle and the injected solution was cast into 8 mm silicone mold, incubated at 37° C., 5% CO₂ for 30 minutes. The hydrogels were cultured up to 7 days and cell viability was assessed using live/dead stain at different time points post injection (30 minutes, days 4 and 7). Rat Schwann cells in saline were also processed similarly and used as controls. The live/dead cell ratio was then analyzed using Image J software.

Statistical analysis: All experiments were performed with n=3 in each group, and data were analyzed using GraphPad Prism. Wherever applicable, the One-Way ANOVA Tukey t test was applied, or unpaired t test was applied to analyze significant differences between the groups. P<0.05 was considered as significant difference between the groups.

### Results:

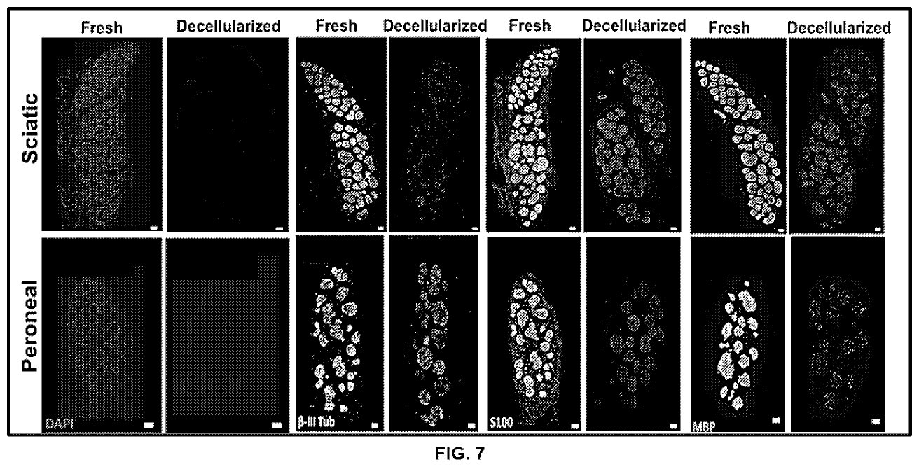

SDD Method Effectively Removes Cells in Decellularized Nerves: Decellularization is the process of efficiently removing cells and cellular membrane remnants while retaining ECM components. The proficiencies of Schwann cell, axon, myelin, and DNA removal were initially analyzed using immunostaining for various markers, S100, β-tubulin III, myelin basic protein (MBP) and DAPI, respectively. As shown in FIG. 7, DAPI staining revealed that both sciatic and peroneal nerves decellularized using the SDD method had no staining for nuclei, as compared to fresh nerves. Similarly, the decellularized nerves had significantly lower staining for β-tubulin III, S100, and MBP, demonstrating efficient removal of axons, Schwann cells, and myelin, respectively. Overall, these results demonstrate that the SDD method can be used to successfully decellularize porcine peripheral (sciatic and peroneal) nerves. Similar results for the SDD method to efficiently remove cells were seen for rat peripheral nerves.

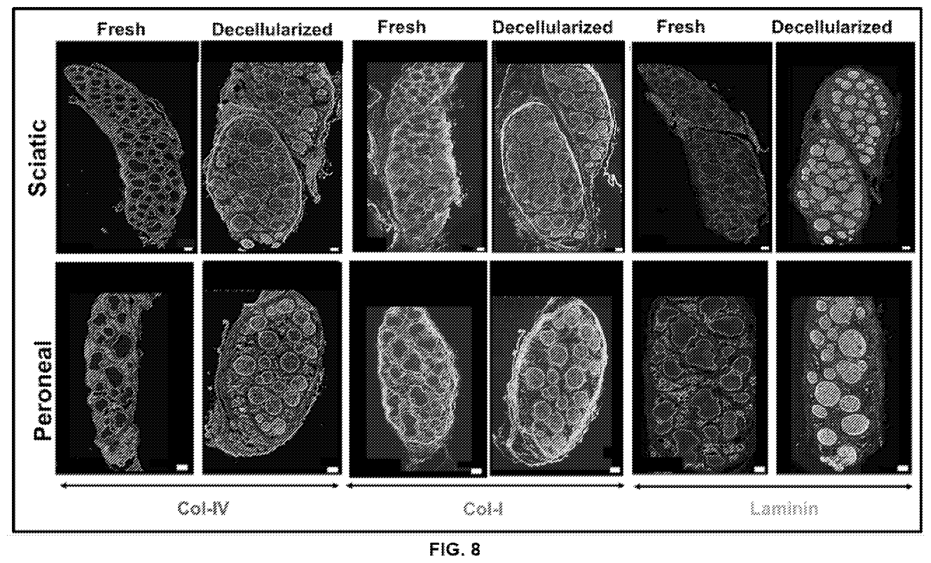

SDD Method Efficiently Retains ECM in Decellularized Nerves: The presence of ECM in decellularized nerves plays an important role in providing pro-regenerative cues for neuronal regeneration. The presence of key ECM Col-I, Col-IV, and laminin were analyzed in porcine sciatic and peroneal decellularized nerves. ECM preservation was also analyzed to ensure that the SDD method retains important ECM constituents in decellularized nerves. As shown in FIG. 8, the SDD method effectively retained and preserved ECM structure in decellularized nerves.

Effects of Residual Lipids on Gelation Kinetics and Mechanical Strength of Hydrogels: The ability of decellularized nerves to undergo gelation at body temperature was further analyzed, which is essential for an in situ gelling, injectable formulation. The SDD method, when previously used to decellularize rat peripheral nerves, resulted in forming an injectable formulation undergoing sol-to-gel transition at body temperature. However, decellularized porcine peripheral nerves failed to undergo efficient sol-to-gel transition, resulting in weak and unstable hydrogels. The effect of lipid remnants in decellularized porcine nerves on the gelation kinetics of hydrogels was studied. To effectively reduce lipid remnants, the decellularized porcine nerves (using SDD method) were subjected to dichloromethane and ethanol solvent for 24 hours and are referred to as “decell+delipid”, whereas only SDD decellularized nerves are addressed as “decell only” in the manuscript.

As shown in FIGS. 4A-9B, dichloromethane and ethanol solvent treatment effectively reduced the lipid remnants in decellularized nerve. These decell+delipid nerves were digested using pepsin solution and neutralized to form a pre-gel solution. Following that, the pre-gel solution was loaded on the rheometer stage at 37° C. and using frequency sweep, G′ and G″ were recorded. G′ values of decell+delipid hydrogels for both sciatic and peroneal samples were higher than values for decell only hydrogels (FIGS. 6A-6F). Furthermore, decell+delipid sciatic and peroneal hydrogels had no increase in G′ after approximately 13.5 minutes, indicating solid hydrogel formation at body temperature. Furthermore, at frequency 1 rad/s, G′ of decell+delipid sciatic and peroneal hydrogels were significantly higher than decell sciatic and peroneal hydrogels only (FIGS. 6A-6F). Therefore, the presence of lipid remnants in decellularized nerves hinders the gelation kinetics of hydrogels and results in lower mechanical strength (lower storage modulus) of hydrogels.

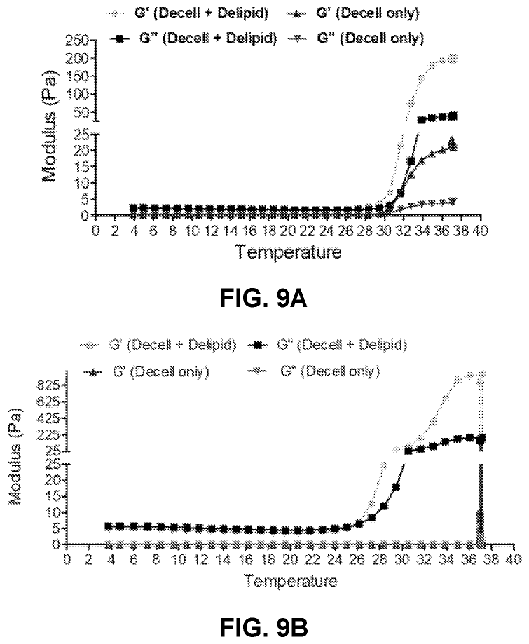

On analyzing the G′ with respect to temperature sweep, it was found that there was no significant difference between decell+delipid and decell only sciatic and peroneal hydrogels. The decell+delipid sciatic hydrogel started to gel at 30° C., as evidenced by a sudden increase in G′ and became stable at 37° C., shown as steady G′ (FIG. 9G). Similarly, for peroneal decell+delipid hydrogels, G′ started to increase at 26° C. while it had stable G′ at 37° C. (FIG. 9H).

Overall, this study indicates that delipidation of porcine peripheral nerves is crucial for forming a stable injectable hydrogel formulation, which is consistent with prior studies showing that delipidation of other fatty tissues improves mechanical strength and gelation kinetics of hydrogels. However, prior research shows that hydrogels with mechanical strength of 2000 Pa would best mimic CNS tissue mechanical strength. Here, it was found that even 10 mg/mL of decell+delipid sciatic and peroneal hydrogels had lower mechanical strength than desired and increasing the concentration of hydrogel solution more than 10 mg/mL would further increase the viscosity of the hydrogel solution, making it difficult to pass through a syringe. Therefore, to achieve a desired mechanical strength of around 2000 Pa, the decell+delipid hydrogels were crosslinked using the natural crosslinking agent genipin.

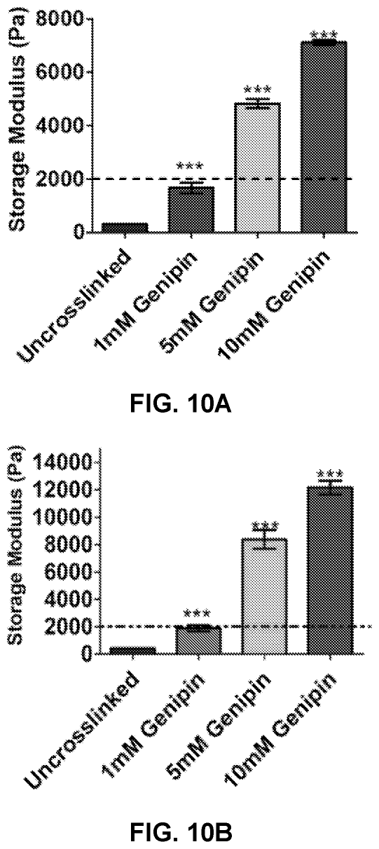

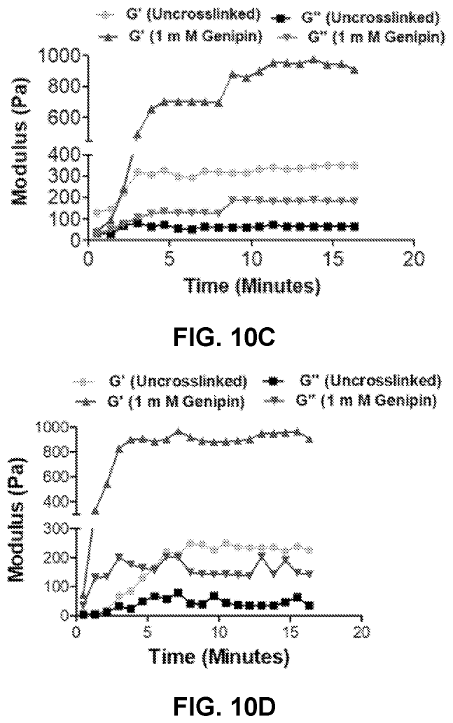

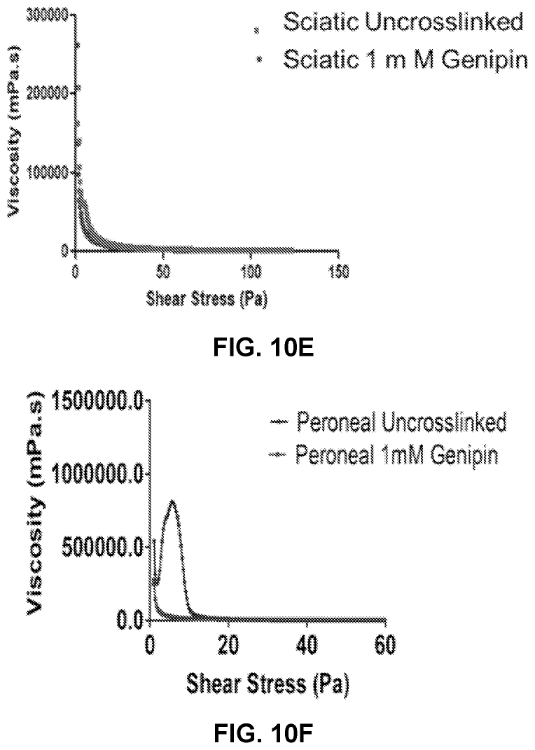

Genipin Crosslinking Tunes Mechanical Strength of Hydrogels and Affects Injectability and Gelation Kinetics: Genipin is a natural and low toxicity crosslinking agent, derived from gardenia fruit, that can crosslink free amino groups of lysine or hydroxylysine residues on different polypeptides chains. Different genipin concentrations (0, 1, 5, 10 mM) on mechanical strength of peroneal and sciatic decell+delipid hydrogels were initially analyzed. To easily differentiate between crosslinked and uncrosslinked hydrogels, “decell+delipid” hydrogels are now referred to as “uncrosslinked”. As shown in FIGS. 10A-10B, an increase in genipin concentration was shown to statistically increase the mechanical strength of hydrogels, as evident by an increase in G′. Uncrosslinked hydrogels that are decell+delipid hydrogels had lower mechanical strength compared to genipin-crosslinked gels, as shown in FIG. 10A. However, as hydrogels of around 2000 Pa are desired for CNS tissue regeneration, sciatic and peroneal decell+delipid hydrogels crosslinked with 1 mM genipin concentration with mechanical strengths of around 2000 Pa were selected for further studies.

The gelation kinetics of both uncrosslinked and 1 mM genipin-crosslinked hydrogel solutions were further analyzed using rheometer time sweep analysis. As shown in FIG. 10C, uncrosslinked decell+delipid sciatic hydrogel started gelling within 2.5 minutes of incubating at 37° C. as evidenced by an increase in G′, while the hydrogel was fully gelled within 15 minutes of incubation at 37° C., with steady G′. The 1 mM genipin crosslinking of decell+delipid sciatic hydrogels had faster gelation kinetics as evident by increase by G′ and after around 10 minutes of incubation at 37° C., the hydrogels were fully gelled as evident by stable G′ (FIG. 10C). Similarly, uncrosslinked decell+delipid peroneal hydrogels started gelling within 7.5 minutes of incubation at 37° C., and fully gelled within 15 minutes. 1 mM genipin crosslinking of decell+delipid peroneal hydrogels speeds the gelation kinetics and G′ starts to increase within 3 minutes and becomes stable within 15 minutes of incubation at 37° C. (FIG. 10D). This study shows that addition of genipin enhances the gelation kinetics of decell+delipid hydrogels and both uncrosslinked and 1 mM genipin-crosslinked sciatic and peroneal hydrogels fully gel within 15 minutes of incubation at 37° C.

On analyzing the injectability of the solution using a rheometer, there was a decrease in viscosity of the solution for both peroneal and sciatic uncrosslinked and 1 mM genipin decell+delipid hydrogels with an increase in shear strain (FIGS. 10E-10F). This demonstrates injectability of the hydrogel as evidenced in previous literature. Furthermore, injectability of peroneal and sciatic decell+delipid uncrosslinked and 1 mM genipin-crosslinked hydrogels was tested using 25-G needles, the most widely used needle for cell delivery. Both uncrosslinked and 1 mM genipin-crosslinked peroneal and sciatic hydrogel solutions were able to pass through syringes easily, demonstrating the suitability of these hydrogel solutions for cell delivery for spinal cord regeneration.

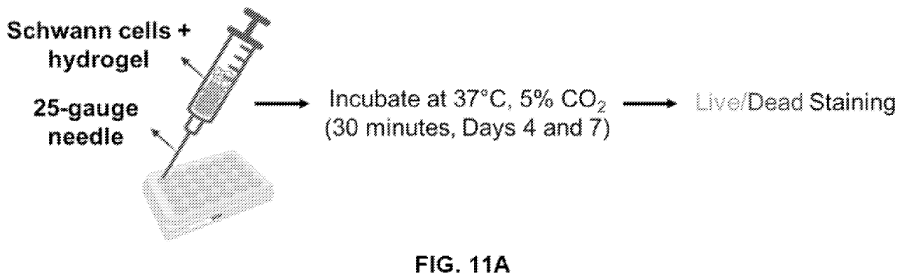

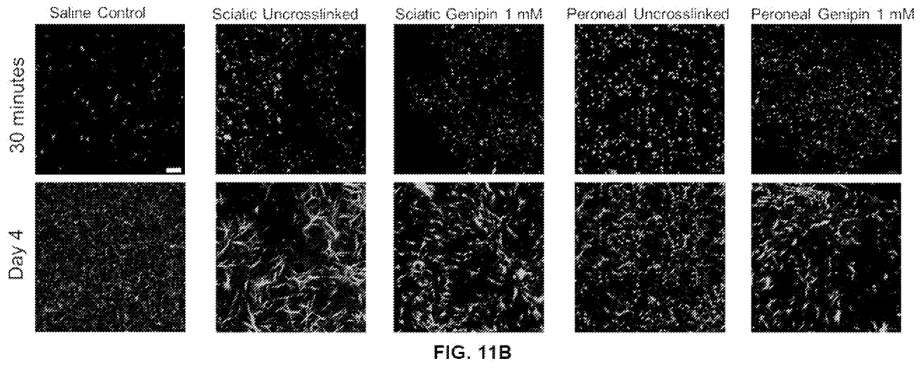

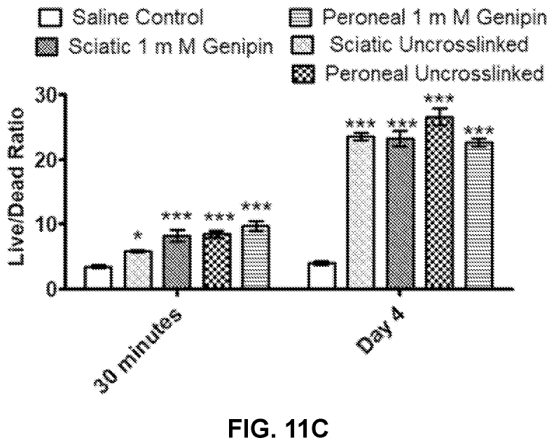

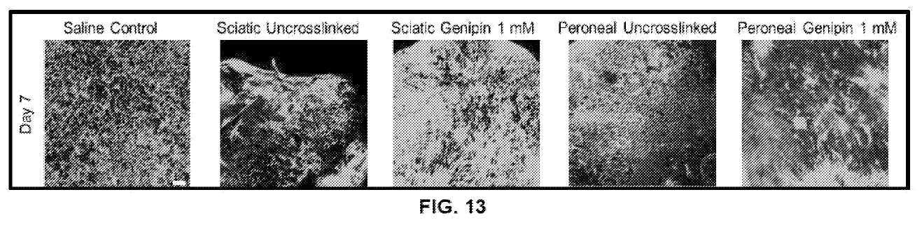

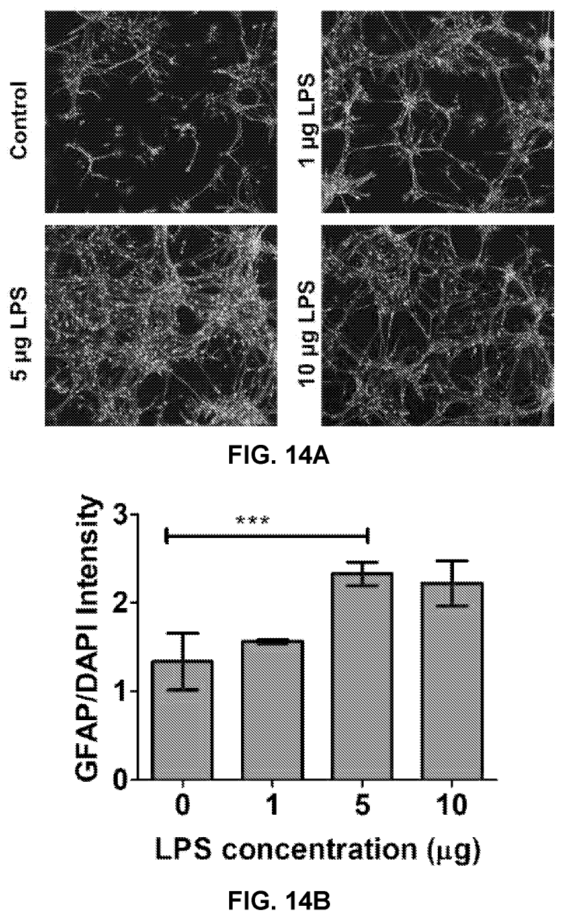

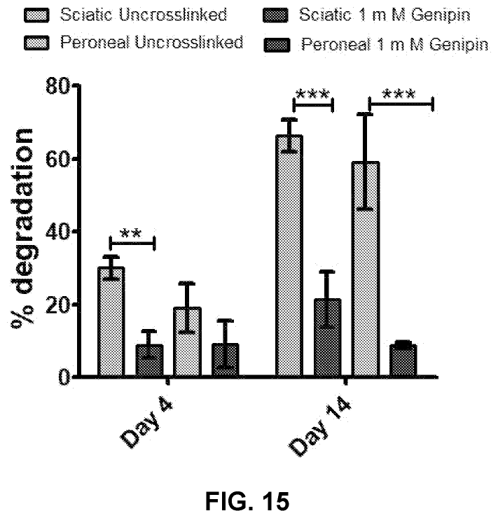

Injectable Hydrogels Maintain Schwann Cell Viability Post Injection: Cell injection procedures typically results in high cell death, with viabilities as low as 1-32% post transplantation, due to mechanical cell membrane disruption during injection and the harsh microenvironment and inflammatory milieu at the site of injury, impacting cell viability. Therefore, Schwann cells viability was initially analyzed post injection using developed injectable hydrogel formulations. Schwann cells were mixed with both uncrosslinked and 1 mM genipin peroneal and sciatic hydrogel formulations and passed through 25-G needles. Schwann cell viability was analyzed post injection at 30 minutes, day 4 and day 7 using live/dead staining (FIG. 11A). As shown in FIGS. 11B-11C, Schwann cells had a statistically higher live/dead cell ratio as compared to saline controls at 30 minutes and day 4 post injection. There was no significant difference between any hydrogel formulations, and both uncrosslinked and 1 mM genipin peroneal and sciatic hydrogel formulations maintained similar cellular viability post injection. However, on day 7 (FIG. 13) there was no difference in live/dead ratios between hydrogel formulations, as compared to saline controls.

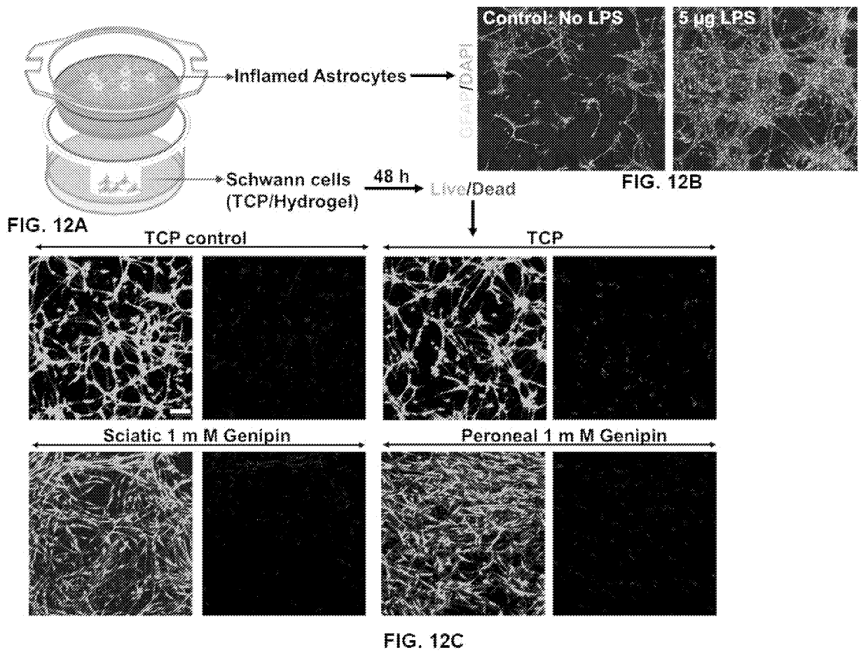

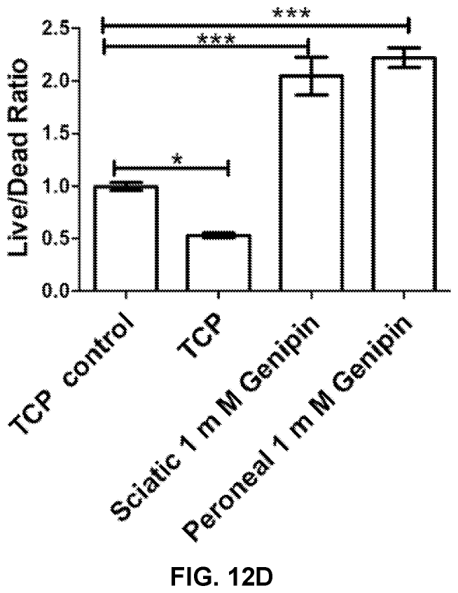

Injectable Hydrogel Maintains Schwann Cell Viability Against Inflammation: After SCI, astrocytes, resident glial cells in the spinal cord, secrete pro-inflammatory cytokines and chemokines, and are proposed as mediators of SCI inflammation and scarring. Therefore, the effects of astrocyte-mediated inflammation on Schwann cell viability were analyzed. Initially, it was determined that the adequate LPS concentration to inflamed astrocytes cells in vitro. Different concentrations of LPS (0, 1, 5, 10 mM) were added to cultured astrocytes and incubated for 24 hours. Following that, GFAP intensity was measured to determine astrocyte reactivity using immunochemistry. Of all LPS concentrations tested, it was found that compared to the no LPS control group, astrocytes treated with 5 mM LPS had higher GFAP intensity, indicating astrocyte reactivity (FIGS. 12A and 13). Thereafter, in co-culture experiments the effect of inflamed astrocytes on Schwann cell viability was assessed. As shown in FIG. 12B, Schwann cells cultured on tissue culture plastic (TCP) in the presence of inflamed astrocytes exhibited higher cell death and a lower live/dead cell ratio. However, Schwann cells encapsulated within decell+delipid peroneal and sciatic 1 mM genipin-crosslinked hydrogels had higher viability and fewer dead cells in the presence of inflamed astrocytes. Therefore, this study indicates that peroneal and sciatic 1 mM genipin-crosslinked hydrogels protect Schwann cells from inflammation-mediated cell death.

### Discussion:

Various methods have previously been studied to decellularize porcine sciatic nerves. However, developing injectable hydrogel formulations using decellularized porcine matrix has remained challenging. Furthermore, the use of decellularized porcine peripheral nerve-based matrices for cell delivery in SCI treatment has not been explored previously. In this study, an injectable hydrogel formulation was developed using decellularized porcine peripheral nerves for increasing the efficiency of Schwann cell transplantation in SCI treatment by maintaining cellular viability during injection and mitigating inflammatory conditions in SCI.

Initially, the potential of a previously developed SDD chemical decellularization method for efficient decellularization of porcine peripheral nerve was evaluated, specifically the sciatic and peroneal branches. The SDD method had previously been successful in efficient decellularization of rat peripheral nerves and fabrication of injectable hydrogel formulations for neural tissue regeneration. After decellularization of porcine peripheral nerves (sciatic and peroneal) using the SDD method, DAPI staining revealed the absence of nuclei, while β III tubulin, S100, and MBP staining revealed the absence of any cellular content including axons, Schwann cells, and myelin debris (FIG. 7). The SDD method also resulted in efficient retention of key ECM components and structure, as evidenced by the presence of Col-I, Col-IV, and laminin staining (FIG. 8). These results indicate the applicability of the SDD method to decellularize porcine peripheral nerves effectively. In addition, these results are consistent with a previous publication, where the SDD method was effective in decellularizing rat peripheral nerves.

Upon using these decellularized matrices for developing injectable hydrogels, it was found that decellularized porcine peripheral nerves formed a weak and unstable injectable hydrogel formulation, unlike prior experiences with rat nerve. Previous literature supports that the presence of lipids in decellularized tissue hinders gelation kinetics of hydrogels, resulting in hydrogels with weak mechanical strength. Besides this, the presence of residual lipids has been shown to inhibit target tissue regeneration by resulting in lower cellular removal efficiency and residual cell debris that can cause immune responses. Similar results were seen in this study: the presence of lipids in decellularized tissues resulted in formation of hydrogels with weak mechanical strength, insufficient for mechanical handling. To reduce lipid remnants in decellularized porcine peripheral nerves, the decellularized tissue was delipidated using dichloromethane and ethanol solvent. Various other solvents like ethanol, 2-propanol, methanol, and acetone have been used previously to reduce lipid remnants for various decellularized tissues. However, dichloromethane and ethanol solvent treatment is most frequently used for reducing lipid remnants in decellularized spinal cord and sciatic nerve tissues. In this study, it was also found that treatment of decellularized tissue using dichloromethane and ethanol solvent significantly reduced lipid remnants and aided in appropriate hydrogel gelation (FIGS. 4A-4B, 6A-6F, and 9A-9B). Therefore, the delipidation of decellularized porcine peripheral nerve tissue is necessary for developing injectable hydrogel formulations with sufficient mechanical strength suitable for injectability.

However, even after delipidation of decellularized porcine peripheral nerves, a 10 mg/mL hydrogel concentration resulted in a mechanical strength less than 2000 Pa. But, for CNS tissue regeneration, it is desired to have hydrogels of around 2000 Pa to mimic CNS tissue mechanical strength. To achieve desired mechanical strength, the decell+delipid hydrogels were crosslinked using a chemical crosslinker, genipin. Genipin is a natural crosslinker that has been widely used by others in the field to crosslink decellularized ECM and genipin-crosslinked decellularized hydrogels have been shown to be biocompatible. In this study, it was found that with an increase in genipin concentration, there was an increase in mechanical strength of hydrogels as evidenced by increases in G′ (FIGS. 10A-10B). The increase in mechanical strength can be attributed to genipin-mediated crosslinking. Genipin crosslinks with the free amino groups of lysine or hydroxylysine residues present on the polypeptides in ECM. As 1 mM genipin crosslinking of decell+delipid hydrogels provided hydrogels of around 2000 Pa mechanical strength, 1 mM genipin-crosslinked hydrogels were used for the remaining studies. Additionally, the decell+delipid hydrogels and both uncrosslinked and 1 mM genipin-crosslinked hydrogels were able to undergo complete gelation within 15 minutes of incubation at 37° C. (FIGS. 10C-10D). Furthermore, crosslinking with 1 mM genipin has been shown to initially reduce the time for gelation of the hydrogels (FIGS. 10C-10D). This gelation time has previously been adequate for biomolecule delivery in SCI animal models.

On analyzing the injectability of the hydrogels using a flow curve rheology study, it was found that both uncrosslinked and genipin-crosslinked decell+delipid hydrogels exhibited a decrease in viscosity with an increase in shear stress. Such shear thinning property of the hydrogels demonstrates that the injectable hydrogel formulations can easily be injected at the target sites without clogging the needles (FIGS. 10E-10F). This was further demonstrated by passing the decell+delipid uncrosslinked and 1 mM genipin-based sciatic and peroneal hydrogel formulations through 25-G needle syringes. This size syringe needle has been used for various cell delivery applications in SCI treatment, and hence, this study demonstrates that the decell+delipid hydrogels are potentially viable options for various clinical cell and biomolecule delivery applications.

In previous pre-clinical animal studies, direct local injection of Schwann cells in saline into the SCI lesion cavity results in significant transplanted cell death. Besides this, regardless of the detrimental host microenvironment, initial cell damage occurs during the actual injection procedure. Cell damage may occur during ejection because of the mechanical disruption of cells. When flowing through a needle, cells experience various types of mechanical forces, including extensional forces and shear forces. Hence, Schwann cell viability was initially assessed when passed through a 25-G needle. It was found that as compared to saline controls, Schwann cells had higher cellular viability in both uncrosslinked and 1 mM genipin sciatic and peroneal injectable hydrogel formulations (FIGS. 11B-11C). Similar results were observed with other injectable hydrogel formulations studied earlier with other cellular types. The increase in cellular viability using injectable hydrogel formulations is a result of the shear protective effect of injectable hydrogel formulations. Next, the effect of the local inflammatory microenvironment on Schwann cell viability when encapsulated within these hydrogels was studied using an in vitro co-culture experiment of Schwann cells and inflamed astrocytes. Astrocytes are resident glial cells in the CNS that secrete pro-inflammatory cytokines and chemokines, mediating SCI inflammation and scarring. Reactive astrocytes have upregulated GFAP expression. The LPS concentration required to activate astrocytes was initially optimized. In this study, it was found that 5 μg of LPS induced astrocytes to significantly upregulate GFAP expression (FIG. 12B), consistent with previous literature. LPS-mediated astrocyte reactivity is mediated by upregulation of Notch signaling, leading to NFkB activation. As genipin-crosslinked hydrogel matched the CNS tissue mechanical strength, and there was no significant difference between uncrosslinked and genipin-crosslinked hydrogels in maintaining cellular viability post injection, only decell+delipid sciatic and peroneal genipin-crosslinked hydrogel formulations were studied to protect Schwann cells against inflamed astrocytes. In the described in vitro inflammatory model, it was found that Schwann cells encapsulated within 1 mM genipin-crosslinked decell+delipid hydrogels (both sciatic and peroneal nerve) had higher cellular viability as compared to Schwann cells cultured on TCP (FIGS. 12C-12D). This study shows that the disclosed fabricated hydrogels protect the Schwann cells from the inflammatory microenvironment to maintain cellular viability and ultimately increase cellular efficacy for potential axonal regeneration in SCI treatment.

### Conclusion:

In this study, a clinically relevant injectable hydrogel formulation was developed using decellularized porcine peripheral nerves (sciatic and peroneal). An SDD-based decellularization method was used to efficiently remove cell nuclear debris and retain important ECM in both sciatic and peroneal decellularized nerves. The presence of lipids in the decellularized porcine nerves was found to hinder the gelation kinetics of the hydrogels, in contrast to prior studies using rat nerve. Thus, a dichloromethane and ethanol solvent treatment was used to efficiently reduce lipid remnants in decellularized porcine nerves, allowing for the fabrication of mechanically stable injectable hydrogels. Delipidation of both sciatic and peroneal decellularized porcine nerves improved gelation kinetics of hydrogels, which would be beneficial for future clinical translation. Furthermore, genipin-mediated chemical crosslinking of both decell+delipid hydrogels (for both sciatic and peroneal) increased the mechanical strength of the hydrogels, and a 1 mM genipin concentration of sciatic and peroneal hydrogel achieved a mechanical strength native to CNS tissue, around 2000 Pa. The decell+delipid hydrogels and 1 mM genipin-crosslinked hydrogels (sciatic and peroneal) both demonstrated shear thinning properties and were able to pass through 25-G needles, which are required for clinical translation. Schwann cells within uncrosslinked and 1 mM genipin decell+delipid hydrogels (sciatic and peroneal), when passed through a 25-G needle, had higher cellular viability as compared to saline controls. In addition, 1 mM genipin-crosslinked hydrogels protected Schwann cells from an astrocyte-mediated inflamed microenvironment. In conclusion, the developed injectable hydrogel formulation holds great potential to efficiently deliver Schwann cells (or other cells) and maintain higher cellular viability.

### Example 3: Human Peripheral Nerve Decellularization

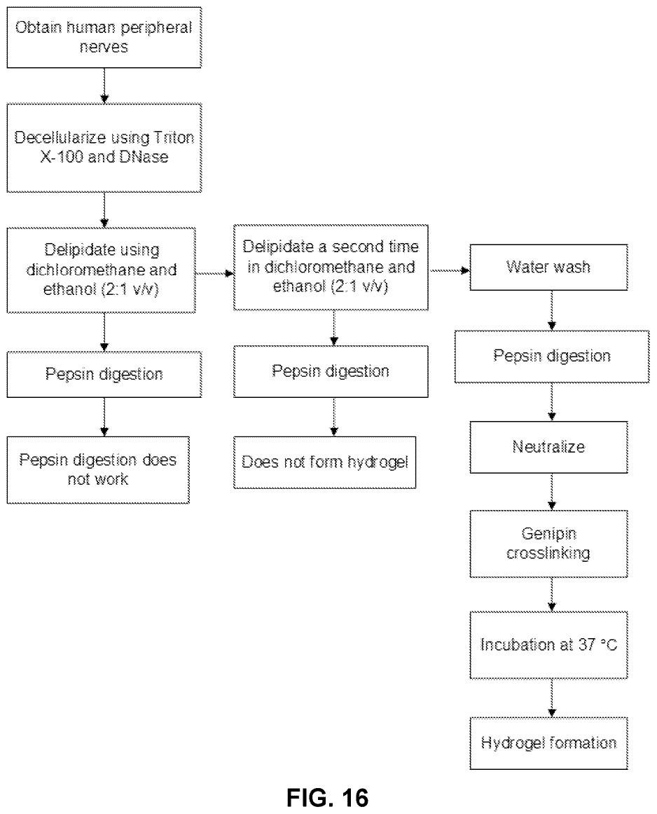

A flow chart describing an exemplary protocol is shown in FIG. 16.

Initially, a previous protocol SDD (sodium deoxycholate-DNase) was attempted to decellularize and form injectable hydrogels from human nerve tissue, however even after successful decellularization, these samples were not able to digest to form injectable hydrogels.

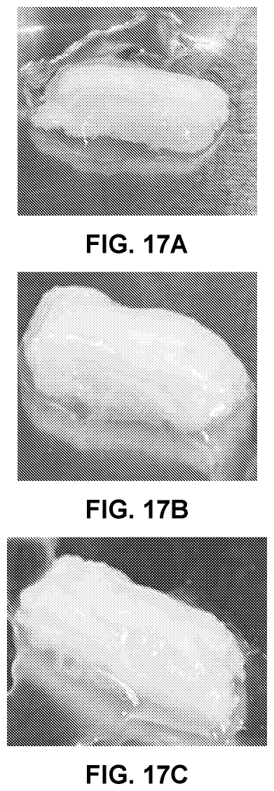

Decellularized human nerves were obtained from Axogen Company and digestion was attempted using pepsin, which worked well. However, the decellularized nerves were not forming hydrogels. The decellularized nerves were subjected to a delipidation treatment using dichloromethane and ethanol solvent, then freeze dried, and digested using pepsin, at which point they formed hydrogels. The same method was then repeated using human nerves not obtained from a commercial source following a previously published decellularization method. In addition, a different solvent hexane:isopropanol (3:2) was explored to determine its effects on delipidation (FIGS. 17A-17C).

The previously published decellularization method was further modified. Triton X-100 was used instead of sodium deoxycholate. Triton X-100 is a non-ionic detergent that disrupts lipid-lipid and lipid-protein interactions while leaving protein-protein interactions intact. A 3 h DNase incubation step was performed in addition to the delipidation step already described. An additional water wash step was introduced at the end of the process to remove bound salts and buffer to decell animals.

The non-commercial nerves digested well, after using this protocol, but the digested solution was not able to form hydrogel when incubated at 37° C. Thus, the crosslinker genipin (5 mM) was used to induce hydrogel formation, which was successful. Even after delipidating these nerve cells, lipid remnants were found in some donors the lipid remnants were still present. Thus, a second round of delipidation was performed using the same solvent (dichloromethane and ethanol in a 2:1 ratio) to see its effect on further reducing lipid remnants.

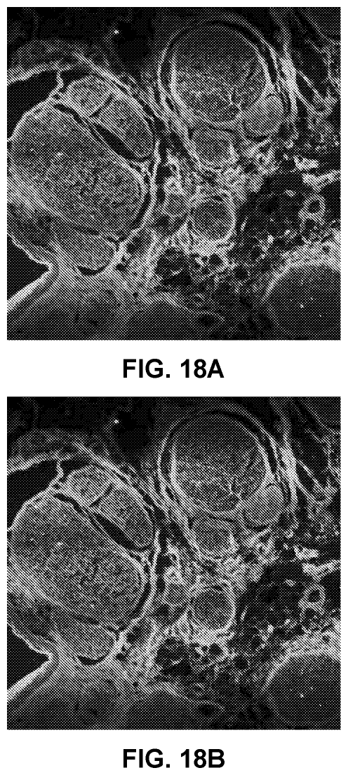

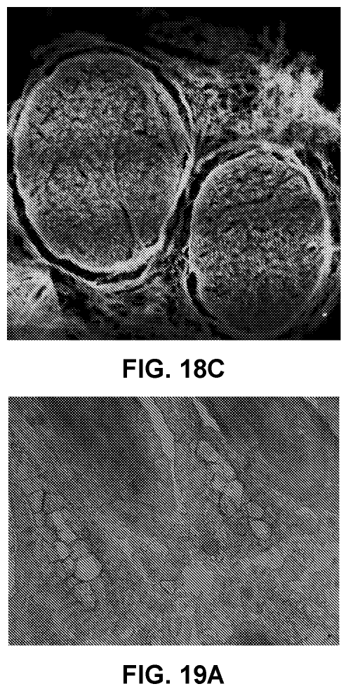

DAPI staining was conducted on fresh nerves, decell nerves, and decell+delipid nerves. Fresh human nerves showed the presence of nuclei (DAPI staining positive), while decell and decell+delipid nerves showed the absence of nuclei. Even after decellularization and delipidation, Collagen-I ECM was found to be retained, as evidenced by the presence of Collagen-I staining (FIGS. 18A-18C). The structure of the ECM may have been damaged due to the use of Triton X-100, however.

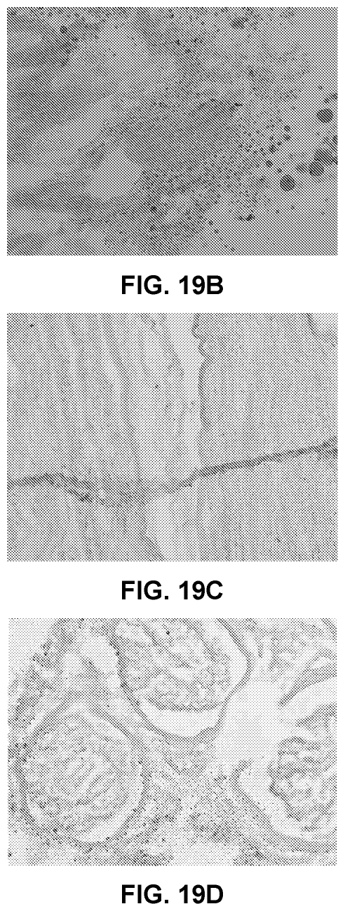

Oil Red O staining was performed as seen in FIGS. 19A-19D. Fresh human nerves show the presence of lipids, while decell samples show lipids present even after Triton X-100 based decellularization. In decell+delipid samples, the lipid was significantly reduced but still present.

However, the digested nerves were still not forming hydrogel without crosslinking and also some donor nerves were not digesting well. A 24-48 hours water wash was introduced to reduce salts and buffer, and this treatment allowed better nerve digestion as well as hydrogel formation.

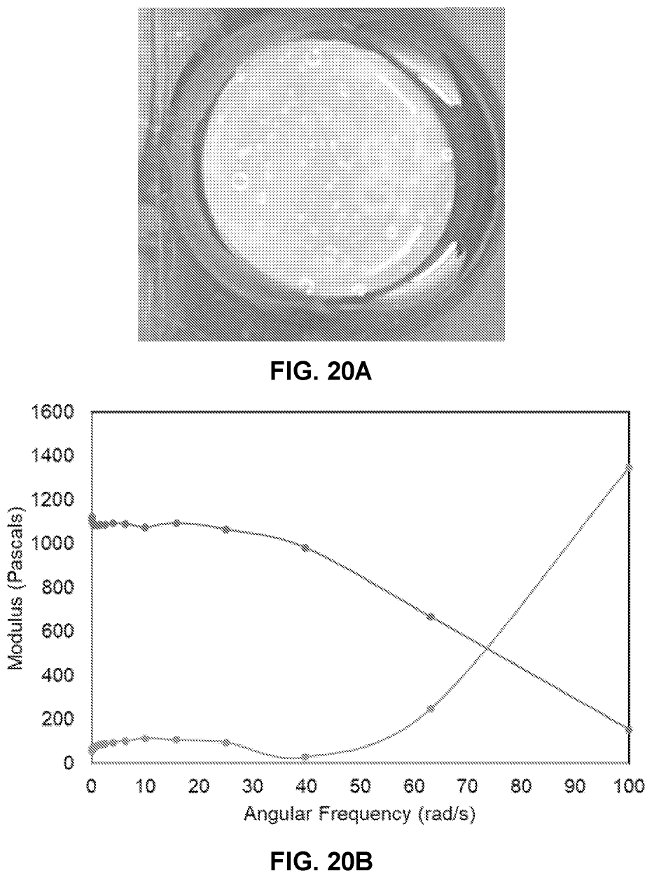

Human decell+delipid nerves digested using pepsin solution were then neutralized using 1 N NaOH solution and 10×PBS. After neutralization, 5 mM genipin was added and hydrogels were cast into silicone molds and incubated at 37° C. for 15-20 min. The hydrogels formed were used for a frequency sweep rheology study (FIGS. 20A-20B).

### REFERENCES

1. Acaz-Fonseca E, et al 2019 Notch signaling in astrocytes mediates their morphological response to an inflammatory challenge Cell Death Discov 5 85
2. Agarwal G, et al 2020 Hyaluronic acid containing scaffolds ameliorate stem cell function for tissue repair and regeneration Int J Biol Macromol 165 388-401
3. Agarwal G, et al 2021 Highly elastic, electroconductive, immunomodulatory graphene crosslinked collagen cryogel for spinal cord regeneration Materials Science and Engineering C 118
4. Agarwal G, et al 2022 Graphene-collagen cryogel controls neuroinflammation and fosters accelerated axonal regeneration in spinal cord injury Biomaterials Advances 139 212971
5. Aguado B A, et al 2012 Improving Viability of Stem Cells During Syringe Needle Flow Through the Design of Hydrogel Cell Carriers Tissue Eng Part A 18 806-15
6. Ahuja C S, et al 2017 Traumatic spinal cord injury Nat Rev Dis Primers 3 17018
7. Almawash S, et al 2022 Current and Future Prospective of Injectable Hydrogels—Design Challenges and Limitations Pharmaceuticals 15 371
8. Alonso J M, et al 2021 Injectable Hydrogels: From Laboratory to Industrialization Polymers (Basel) 13 650
9. Amer M H, et al 2017 Translational considerations in injectable cell-based therapeutics for neurological applications: concepts, progress and challenges NPJ Regen Med 2 23
10. Bhuiyan M H, et al 2023 Optimization of thermoresponsive chitosan/β-glycerophosphate hydrogels for injectable neural tissue engineering application Colloids Surf B Biointerfaces 224 113193
11. Bidarra S J, et al 2014 Injectable alginate hydrogels for cell delivery in tissue engineering Acta Biomater 10 1646-62
12. Bousalis D, et al 2022 Decellularized peripheral nerve as an injectable delivery vehicle for neural applications J Biomed Mater Res A 110 595-611
13. Brown M, et al 2022 Decellularized extracellular matrix: New promising and challenging biomaterials for regenerative medicine Biomaterials 289 121786
14. Cerqueira S R, et al 2018 Decellularized peripheral nerve supports Schwann cell transplants and axon growth following spinal cord injury Biomaterials 177 176-85
15. Chen S, et al 2019 Promoting Neurite Growth and Schwann Cell Migration by the Harnessing Decellularized Nerve Matrix onto Nanofibrous Guidance ACS Appl Mater Interfaces 11 17167-76
16. Cheng J, et al 2023 Curcumin protects from LPS-induced activation of astrocytes via AMPK pathway Neuroreport 34 748-58
17. Choi J, et al 2018 Decellularized sciatic nerve matrix as a biodegradable conduit for peripheral nerve regeneration. Neural Regen Res 13 1796-803
18. Cornelison R C, et al 2018 Injectable hydrogels of optimized acellular nerve for injection in the injured spinal cord Biomedical Materials 13 34110
19. Crapo P M, et al 2011 An overview of tissue and whole organ decellularization processes Biomaterials 32 3233-43
20. Davaa G, et al 2023 Delivery of Induced Neural Stem Cells Through Mechano-Tuned Silk-Collagen Hydrogels for the Recovery of Contused Spinal Cord in Rats Adv Healthc Mater 12
21. Ding X, et al 2021 Injectable In Situ Forming Double-Network Hydrogel To Enhance Transplanted Cell Viability and Retention Chemistry of Materials 33 5885-95
22. Du A, et al 2022 Genipin-crosslinked decellularized scaffold induces regeneration of defective rat kidneys J Biomater Appl 37 415-28
23. Fan B, et al 2022 Progression in translational research on spinal cord injury based on microenvironment imbalance Bone Res 10 35
24. Fu Y, et al 2016 Decellularization of porcine skeletal muscle extracellular matrix for the formulation of a matrix hydrogel: a preliminary study J Cell Mol Med 20 740-9
25. Gant K L, et al 2022 Phase 1 Safety Trial of Autologous Human Schwann Cell Transplantation in Chronic Spinal Cord Injury J Neurotrauma 39 285-99
26. Gardin C, et al 2015 Decellularization and Delipidation Protocols of Bovine Bone and Pericardium for Bone Grafting and Guided Bone Regeneration Procedures PLoS One 10 e0132344
27. Hansson E, et al 2016 Therapeutic innovation: Inflammatory-reactive astrocytes as targets of inflammation IBRO Rep 1 1-9
28. Hernandez, et al. 2018 Decellularized Extracellular Matrix Hydrogels as a Delivery Platform for MicroRNA and Extracellular Vesicle Therapeutics, Adv. Ther (Weinh); 1(3) 1800032
29. Hill C E, et al 2007 Early necrosis and apoptosis of Schwann cells transplanted into the injured rat spinal cord European Journal of Neuroscience 26 1433-45
30. Hlavac N, et al 2020 Progress toward finding the perfect match: hydrogels for treatment of central nervous system injury Mater Today Adv 6 100039
31. Huang H, et al 2022 Advances and prospects of cell therapy for spinal cord injury patients Journal of Neurorestoratology 10 13-30
32. Hudson T W, et al 2004 Optimized Acellular Nerve Graft Is Immunologically Tolerated and Supports Regeneration Tissue Eng 10 1641-51
33. Kasper M, et al 2020 Bench-to-Bedside Lessons Learned: Commercialization of an Acellular Nerve Graft Adv Healthc Mater 9
34. Kim B S, et al 2020 Decellularized Extracellular Matrix-based Bioinks for Engineering Tissue- and Organ-specific Microenvironments Chem Rev 120 10608-61
35. Kong Y, et al 2022 Electrospinning porcine decellularized nerve matrix scaffold for peripheral nerve regeneration Int J Biol Macromol 209 1867-81
36. Kwiecien J M, et al 2020 Prolonged inflammation leads to ongoing damage after spinal cord injury PLoS One 15 e0226584
37. Lee J H 2018 Injectable hydrogels delivering therapeutic agents for disease treatment and tissue engineering Biomater Res 22 27
38. Li T, et al 2020 Fabrication and Evaluation of a Xenogeneic Decellularized Nerve-Derived Material: Preclinical Studies of a New Strategy for Nerve Repair Neurotherapeutics 17 356-70
39. Li Y, et al 2022 Biodegradable and Injectable Hydrogels in Biomedical Applications Biomacromolecules 23 609-18
40. Lin T, et al 2018 Hydrogel derived from porcine decellularized nerve tissue as a promising biomaterial for repairing peripheral nerve defects Acta Biomater 73 326-38
41. Marquardt L M, et al 2020 Designer, injectable gels to prevent transplanted Schwann cell loss during spinal cord injury therapy Sci Adv 6
42. McCrary M W, et al 2020 Novel Sodium Deoxycholate-Based Chemical Decellularization Method for Peripheral Nerve Tissue Eng Part C Methods, 2020, 23-36.
43. Meder T, et al 2021 Nerve-specific extracellular matrix hydrogel promotes functional regeneration following nerve gap injury NPJ Regen Med 6 69
44. Mendibil U, et al 2020 Tissue-Specific Decellularization Methods: Rationale and Strategies to Achieve Regenerative Compounds Int J Mol Sci 21 5447
45. Min K, et al 2022 Three-Dimensional Microfilament Printing of a Decellularized Extracellular Matrix (dECM) Bioink Using a Microgel Printing Bath for Nerve Graft Fabrication and the Effectiveness of dECM Graft Combined with a Polycaprolactone Conduit ACS Appl Bio Mater 5 1591-603
46. Mohammadi F, et al 2022 Perception of facing life's challenges in patients with spinal cord injury in Iran: a qualitative study BMC Psychol 10 202
47. Monje P V., et al 2021 Human Schwann Cell Transplantation for Spinal Cord Injury: Prospects and Challenges in Translational Medicine Front Cell Neurosci 15
48. Nishiguchi A, et al 2021 A pH-driven genipin gelator to engineer decellularized extracellular matrix-based tissue adhesives Acta Biomater 131 211-21
49. Oraee-Yazdani S, et al 2021 Combining cell therapy with human autologous Schwann cell and bone marrow-derived mesenchymal stem cell in patients with subacute complete spinal cord injury: safety considerations and possible outcomes Stem Cell Res Ther 12 445
50. Pearse D D, et al 2007 Transplantation of Schwann cells and/or olfactory ensheathing glia into the contused spinal cord: Survival, migration, axon association, and functional recovery Glia 55 976-1000
51. Piantanida E, et al 2019 Design of Nanocomposite Injectable Hydrogels for Minimally Invasive Surgery Acc Chem Res 52 2101-12
52. Qin C, et al 2018 Induced Pluripotent Stem Cell Transplantation Improves Locomotor Recovery in Rat Models of Spinal Cord Injury: a Systematic Review and Meta-Analysis of Randomized Controlled Trials Cellular Physiology and Biochemistry 47 1835-52
53. Rao Z, et al 2021 Decellularized nerve matrix hydrogel scaffolds with longitudinally oriented and size-tunable microchannels for peripheral nerve regeneration Materials Science and Engineering: C 120 111791
54. Sackett S D, et al 2018 Extracellular matrix scaffold and hydrogel derived from decellularized and delipidized human pancreas Sci Rep 8 10452
55. Seif-Naraghi S B, et al 2010 Design and Characterization of an Injectable Pericardial Matrix Gel: A Potentially Autologous Scaffold for Cardiac Tissue Engineering Tissue Eng Part A 16 2017-27
56. Shang Z, et al 2022 What Is the Optimal Timing of Transplantation of Neural Stem Cells in Spinal Cord Injury?A Systematic Review and Network Meta-Analysis Based on Animal Studies Front Immunol 13
57. Srikandarajah N, et al 2023 Current insights into the management of spinal cord injury J Orthop 41 8-13
58. Tukmachev D, et al 2016 Injectable Extracellular Matrix Hydrogels as Scaffolds for Spinal Cord Injury Repair Tissue Eng Part A 22 306-17
59. Vyborny K, et al 2019 Genipin and EDC crosslinking of extracellular matrix hydrogel derived from human umbilical cord for neural tissue repair Sci Rep 9 10674
60. Wachs R A, et al 2023 Apoptosis-Decellularized Peripheral Nerve Scaffold Allows Regeneration across Nerve Gap Cells Tissues Organs 212 512-22
61. Wang Y, et al 2016 Genipin crosslinking reduced the immunogenicity of xenogeneic decellularized porcine whole-liver matrices through regulation of immune cell proliferation and polarization Sci Rep 6 24779
62. Zhang J, et al 2016 Allogeneic adipose-derived stem cells promote survival of fat grafts in immunocompetent diabetic rats Cell Tissue Res 364 357-67
63. Zhang W, et al 2021 Research progress in decellularized extracellular matrix-derived hydrogels Regen Ther 18 88-96
64. Zheng C, et al 2021 Nanofibrous nerve guidance conduits decorated with decellularized matrix hydrogel facilitate peripheral nerve injury repair Theranostics 11 2917-31
65. Zipser C M, et al 2022 Cell-based and stem-cell-based treatments for spinal cord injury: evidence from clinical trials Lancet Neurol 21 659-70

1.-25. (canceled)

26. A method comprising:
administering a tissue hydrogel to a subject;
wherein the tissue hydrogel comprises decellularized, delipidated tissue comprising native extracellular matrix proteins,
wherein the decellularized, delipidated tissue is cross-linked to form the hydrogel.

27. The method of claim 26, further comprising co-administering a population of cells with the tissue hydrogel.

28. The method of claim 26, wherein the subject has a peripheral nerve injury, spinal cord injury, spinal cord disease, amputation, has degradation of a component of the spine, osteoarthritis, lung trauma, volumetric muscle loss, or liver cirrhosis.

29.-67. (canceled)

68. The method of claim 26, further comprising co-administering a therapeutic agent with the tissue hydrogel.

69. The method of claim 68, wherein the therapeutic agent is a polynucleotide, an amino acid, a peptide, a polypeptide, an antibody, an aptamer, a ribozyme, a guide sequence for a ribozyme that inhibit translation or transcription of essential tumor proteins and genes, a hormone, an immunomodulator, an antipyretic, an anxiolytic, an antipsychotic, an analgesic, an antispasmodic, an anti-inflammatory agent, an anti-histamine, an anti-infective, a chemotherapeutic, or a combination thereof.

70. The method of claim 26, wherein the decellularized, delipidated tissue is decellularized, delipidated brain, spinal cord, nerve, heart, lung, liver, muscle, cartilage, tendons, ligament, menisci, stomach, intestine, pancreas, kidney, blood vessel, bone, or cornea tissue.

71. The method of claim 70, wherein the decellularized, delipidated tissue is nervous tissue from a mammal selected from the group consisting of: pig, bovine, and human.

72. The method of claim 26, wherein the decellularized, delipidated tissue hydrogel is formed by the method comprising:
(a) removing cells and DNA from a tissue sample from a donor with a decellularization process that retains native extracellular matrix (ECM) components to produce a decellularized tissue;
(b) removing lipids from the decellularized tissue from (a) with a delipidation process that retains the native ECM components to produce a decellularized, delipidated tissue;
(c) enzymatically digesting the decellularized, delipidated tissue from (b); and
(d) neutralizing the digested, decellularized, delipidated tissue from (c) to form a pre-gel solution; and
(e) exposing the pre-gel solution to temperatures of about 37° C. for an amount of time to form the tissue hydrogel.

73. The method of claim 72, wherein the decellularization process comprises:
(i) inducing widespread apoptosis in the tissue sample;
(ii) removing apoptotic bodies using a mild hyper or hypo tonic buffered solution; and
(iii) removing DNA by exposing the tissue from (ii) to DNase.

74. The method of claim 72, wherein the decellularization process comprises:
(i) incubating the tissue sample in a solution containing sodium deoxycholate or Triton X-100;
(ii) removing DNA from the tissue from (i) by exposing the tissue from (i) to DNase; and
(iii) washing the tissue from (ii) in phosphate-buffered saline (PBS).

75. The method of claim 74, wherein the solution in step (i) also includes SB-10, SB-16, or a combination thereof.

76. The method of claim 72, wherein the delipidation process comprises:
(i) incubating the decellularized tissue from (a) with a delipidation solvent for an amount of time to remove lipids; and
(ii) washing the tissue from (i) to remove residual lipids.

77. The method of claim 76, wherein the delipidation solvent is selected from the group consisting of: a combination of ethanol and dichloromethane; a combination of chloroform and methanol; a combination of hexane and isopropanol; and type I water.

78. The method of claim 77, wherein the delipidation process is repeated a second time and wherein the delipidation solvent for the repeated delipidation process is the same as the delipidation solvent in step (i).

79. The method of claim 77, wherein the delipidation solvent comprises a combination of ethanol and dichloromethane in a ratio of from about 1:1 to about 1:4.

80. The method of claim 77, wherein the delipidation solvent comprises a combination of hexane and isopropanol in a ratio of about 3:2.

81. The method of claim 72, wherein step (e) is performed in vivo.

82. The method of claim 72, wherein step (c) is performed by incubating the tissue from step (b) in a solution comprising pepsin.

83. The method of claim 72, further comprising lyophilizing the tissue from step (b) before step (c).

84. The method of claim 72, further comprising washing the tissue hydrogel with water to remove bound salts and buffers.
